(19) United States
(12) Patent Application Publication
(10) Pub. No.: US 2023/0389776 A1
(43) Pub. Date: Dec. 7, 2023

(54) CONDUIT SWITCHING DEVICE AND ENDOSCOPE SYSTEM

(71) Applicant: OLYMPUS MEDICAL SYSTEMS CORP., Tokyo (JP)

(72) Inventors: Takumi KAWAKAMI, Tokyo (JP); Hiroshi TSURUTA, Sagamihara-shi (JP); Naoyuki FUJITA, Tokyo (JP); Eijiro SATO, Tokyo (JP)

(73) Assignee: OLYMPUS MEDICAL SYSTEMS CORP., Tokyo (JP)

(21) Appl. No.: 18/205,588

(22) Filed: Jun. 5, 2023

Related U.S. Application Data
(60) Provisional application No. 63/349,210, filed on Jun. 6, 2022.

(51) Int. Cl. A61B 1/00; A61B 1/015

(52) U.S. Cl. CPC A61B 1/00068; A61B 1/015; A61B 1/00137; A61B 1/00094; A61B 1/00119

(57) ABSTRACT

A conduit switching device includes a body, an endoscope connection port, a fluid supply port, a fluid suction port, a first conduit in the body and the fluid supply port, a second conduit in the body and the fluid suction port, a common conduit in the body and the endoscope connection port, and a switching mechanism configured to be switched between a first state and a second state. In the first state, the first conduit communicates with the common conduit, and the second conduit does not communicate with the common conduit. In the second state, the first conduit does not communicate with the common conduit, and the second conduit communicates with the common conduit.

### RELATED APPLICATION DATA

This application is based on and claims priority under 37 U.S.C. § 119 to U.S. Provisional Application No. 63/349,210 filed on Jun. 6, 2022, the entire contents of which are incorporated herein by reference.

### FIELD OF DISCLOSURE

The present disclosure relates to a conduit selection device that can select a fluid supply conduit for liquid feeding or a fluid outflow conduit for suction, and to an endoscope apparatus that includes the conduit selection device.

### BACKGROUND

For example, an endoscope for urinary organ is inserted into a target part (a renal pelvis, a calyx, a ureter, a bladder, or a urethra, for example) of a subject.

There may be cases in which the endoscope inserted into the target part has a liquid feeding function and a suction function in order to collect foreign substances, such as stones, from the target part while ensuring a field of view in the target part.

Therefore, a port of an endoscope conduit that is provided in an operation portion of the endoscope is connected to an external liquid feeding source to supply fluid to a distal end portion of the endoscope, or is connected to an external suction device to suction fluid from the distal end portion.

Japanese Patent Application Laid-Open Publication No. 2008-200372, for example, discloses a configuration in which when a valve operation portion is rotated, a valve member is sequentially switched through a number of positions.

The positions to which the valve member is switched include a communication cut-off position, at which a connection port is cut off from a supply port and a suction port, a first connection position, at which the connection port is connected to the supply port, positions at which the respective ports communicate with each other or cut off from each other, and a second connection position, at which the connection port is connected to the suction port.

When the valve operation portion is positioned at intervals of a predetermined angle in one direction and is rotated, the valve member is switched in order or in reverse order of the above-described respective positions.

However, the suction function is not always used in all procedures. For this reason, two products, that is, a first endoscope having no suction function and a second endoscope having the suction function, are prepared for a cystoscope, for example.

The first endoscope includes one endoscope conduit, an operation portion has one opening, and a liquid feeding conduit is connected to the one opening.

In the second endoscope, one endoscope conduit branches into two conduits in an operation portion, and an operation portion has two openings that correspond to the respective branch conduits.

A liquid feeding conduit is connected to one opening, and a suction conduit is connected to the other opening.

### SUMMARY

A conduit selection device according to one aspect of the present disclosure includes: a body, an endoscope connection port, a fluid supply port, a fluid suction port, a first conduit in the body and the fluid supply port, a second conduit in the body and the fluid suction port, a common conduit in the body and the endoscope connection port, and a switching mechanism configured to be switched between a first state and a second state. In the first state, the first conduit communicates with the common conduit, and the second conduit does not communicate with the common conduit. In the second state, the first conduit does not communicate with the common conduit, and the second conduit communicates with the common conduit.

### BRIEF DESCRIPTION OF THE DRAWINGS

FIG. 1 is a diagram showing one configuration example of an endoscope apparatus of a first embodiment of the present disclosure.

FIG. 2 is a diagram showing another configuration example of the endoscope apparatus of the first embodiment.

FIG. 3 is a diagram showing a configuration example of a conduit selection device of the first embodiment.

FIG. 4 is a diagram showing an example in which a joint part is used in mounting a conduit selection device of a second embodiment of the present disclosure on an endoscope.

FIG. 5 is a table showing examples of a joint part that corresponds to a dominant hand of an operator in the second embodiment.

FIG. 6 is a table showing a configuration example of a selection mechanism of a conduit selection device of a third embodiment of the present disclosure.

FIG. 7 is a table showing a configuration example of a selection mechanism of a conduit selection device of a fourth embodiment of the present disclosure.

FIG. 8 is a table showing a configuration example of a selection mechanism of a conduit selection device of a fifth embodiment of the present disclosure.

FIG. 9 is a table showing a schematic configuration example of a selection mechanism of a conduit selection device of a sixth embodiment of the present disclosure.

FIG. 10 is a table schematically showing a knock cam mechanism of the selection mechanism in the sixth embodiment in an enlarged manner.

FIG. 11 is a perspective view showing an external appearance of a conduit selection device of a seventh embodiment of the present disclosure.

FIG. 12 is a table showing a configuration example of a selection mechanism of the conduit selection device of the seventh embodiment.

FIG. 13 is a diagram showing a configuration example of a conduit selection device of an eighth embodiment of the present disclosure.

FIG. 14 is a perspective view showing a configuration example of a selection mechanism of the conduit selection device of the eighth embodiment.

FIG. 15 is a perspective view showing a configuration example of a rotary plate of the selection mechanism in the eighth embodiment as viewed from a cam side.

FIG. 16 is a diagram showing the rotary plate disposed on a surface of a body of the conduit selection device of the eighth embodiment.

FIG. 17 is a table showing configuration examples of a first conduit switching plate, a second conduit switching plate, and a third conduit switching plate of the conduit selection device of the eighth embodiment as viewed in an oblique direction.

FIG. 18 is a table showing configuration examples of a conduit selection device of a modification of the eighth embodiment.

FIG. 19 is a diagram showing a configuration example of a conduit selection device of a ninth embodiment of the present disclosure.

FIG. 20 is a table illustrating a state of switching the conduit selection device of the ninth embodiment between a first state and a second state.

FIG. 21 is a diagram showing an example of arrangement of a first conduit, a second conduit, and an air suction conduit in a conduit selection device of a tenth embodiment of the present disclosure.

FIG. 22 is a table showing a configuration example of the conduit selection device of the tenth embodiment.

FIG. 23 is a table showing a configuration example of a conduit selection device of an eleventh embodiment of the present disclosure.

FIG. 24 is a table showing a configuration example of a conduit selection device of a twelfth embodiment of the present disclosure.

FIG. 25 is a table showing a configuration example of a conduit selection device of a thirteenth embodiment of the present disclosure.

FIG. 26 is a table showing a configuration example of a conduit selection device of a fourteenth embodiment of the present disclosure.

FIG. 27 is a table showing configuration examples of a first conduit switching plate, a second conduit switching plate, and a third conduit switching plate of the conduit selection device of the fourteenth embodiment.

### DETAILED DESCRIPTION

In general, preparing a plurality of kinds of endoscope increases an introduction cost for a medical institution, and requires a manufacturer to use a plurality of manufacturing lines, thus increasing manufacturing costs.

Provided that the other opening is closed, a second endoscope having a suction function can also be used in cases in which the suction function is not used and hence, it can be considered that the manufacturer manufactures only the second endoscopes, and the medical institution introduces only the second endoscopes.

However, in the second endoscope, two branch conduits communicate with each other at a branch portion.

For this reason, even in cases in which the suction function is not used, the inside of the branch conduit communicating with the closed other opening is cleaned after a surgical operation.

As a result, a management cost increases for the medical institution.

Further, also with regard to manufacturers, the second endoscopes require a higher manufacturing cost than first endoscopes having no suction function.

There is a three-way cock known as a device that can switch conduits.

Therefore, it is possible to consider that a three-way cock is connected to a first endoscope to switch conduits.

However, the three-way cock is generally connected via a tube, thus being disposed at a position away from an operation portion.

For this reason, it is difficult for an operator to directly perform an operation of switching the three-way cock close to hand.

According to embodiments described hereinafter, it is possible to provide a conduit selection device and an endoscope system that allow the operator grasping an endoscope to switch conduits close to hand with excellent ease of operation, thus eliminating requirement for preparing an endoscope including an endoscope conduit with branches, leading to a reduction in costs.

Hereinafter, embodiments of the present disclosure will be described with reference to drawings.

However, the present disclosure is not limited by the embodiments described hereinafter.

In the drawings, identical or corresponding elements are given the same reference symbols when appropriate.

The drawings are schematic views. Note that, in one drawing, relationships of lengths of the respective elements, ratios between the lengths of the respective elements, and the number of the respective elements, for example, may differ from actual ones in order to simplify the description.

The relationship or the ratio between the lengths may be partially different between the plurality of drawings.

### First Embodiment

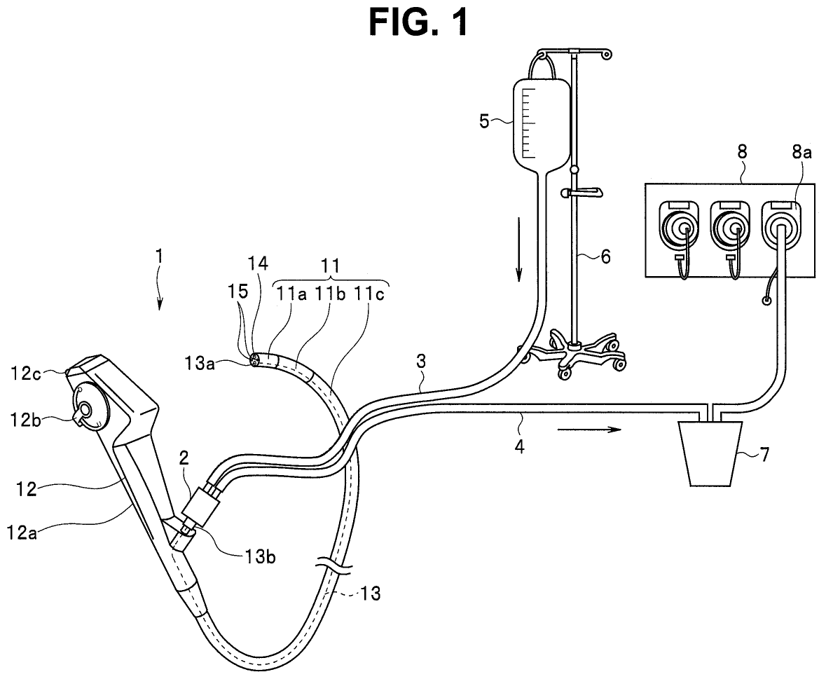

FIG. 1 to FIG. 3 show a first embodiment of the present disclosure.

FIG. 1 is a diagram showing one configuration example of an endoscope apparatus of the first embodiment.

As shown in FIG. 1, the endoscope apparatus of the present embodiment includes an endoscope 1, a conduit selection device (conduit switching device) 2, a liquid feeding tube 3, a suction tube 4, a liquid feeding source 5, a caster 6, a waste liquid container 7, and a suction device 8.

The endoscope 1 is a device used for observing a subject or for performing treatment on the subject.

The endoscope 1 includes an insertion portion 11 and an operation portion 12, the insertion portion 11 being to be inserted into the subject, the operation portion 12 being provided at a proximal end side of the insertion portion 11.

Assume that the subject into which the insertion portion 11 is inserted is a living body, such as a human or an animal.

Examples of a target part for inspection include a kidney (a renal pelvis, a calyx, or the like) and a urinary tract, such as a ureter, a bladder, and a urethra.

Accordingly, one example of the endoscope 1 includes a ureteroscope.

The endoscope 1 may be a reusable endoscope that can be used a plurality of times by performing reprocessing treatment on the endoscope 1, or may be a single-use endoscope that is disposed of (discarded) after being used one time.

The insertion portion 11 includes a distal end portion 11a, a bending portion 11b, and a tubular portion 11c in order from a distal end toward a proximal end of the insertion portion 11.

The distal end portion 11a includes an observation system 14 and an illumination system 15.

The observation system 14 forms an optical image of the subject with an objective optical system, and photoelectrically converts the optical image with an image pickup device to generate an image pickup signal.

The illumination system 15 transmits illumination light with a light guide, for example, to irradiate the subject with the illumination light from a distal end of the light guide.

A signal line, which is connected to the image pickup device, and the light guide are connected to an endoscope control device not shown in the drawing via the insertion portion 11, the operation portion 12, and a universal cable not shown in the drawing.

The endoscope control device controls the endoscope 1, and processes an image pickup signal obtained from the endoscope 1.

The endoscope control device also serves as an image processing device and a light source device, for example, and supplies illumination light to the endoscope 1.

The endoscope control device performs image processing on an image pickup signal to generate an image signal, and causes a monitor connected to the endoscope control device to display an endoscope image.

The bending portion 11b is provided at a proximal end side of the distal end portion 11a, and is configured to be bendable in two directions or four directions of an upward direction, a downward direction, a leftward direction, and a rightward direction, for example.

When the bending portion 11b is bent, a direction of the distal end portion 11a changes, so that a direction of observation by the observation system 14 and a direction in which the illumination system 15 performs irradiation with illumination light change.

The bending portion 11b is also bent to increase ease of insertion of the insertion portion 11 in the subject.

The tubular portion 11c is a tubular part that couples a proximal end of the bending portion 11b to a distal end of the operation portion 12.

The tubular portion 11c may be a rigid tubular portion that prevents the insertion portion 11 from deflecting, or may be a flexible tubular portion that allows the insertion portion 11 to deflect in conformity with a shape of the subject into which the insertion portion 11 is inserted.

An endoscope that includes a rigid insertion portion is generally referred to as a rigid endoscope, and an endoscope that includes a flexible insertion portion is generally referred to as a flexible endoscope.

For example, a rigid endoscope and a flexible endoscope in a medical field are defined in ISO 8600-1: 2015.

An endoscope conduit 13 is provided to extend from an inside of the distal end portion 11a of the insertion portion 11 to a portion of the operation portion 12, and liquid feeding and suction is performed through the endoscope conduit 13.

The endoscope conduit 13 has a distal-end-side opening 13a formed in a distal end surface of the distal end portion 11a, and has a proximal-end-side opening 13b formed in a distal end side of the operation portion 12.

The endoscope conduit 13 also serves as a treatment instrument conduit, for example, and allows a treatment instrument to be inserted through the endoscope conduit 13.

Some examples of the treatment instrument include a laser probe for crushing stones, and forceps.

The endoscope 1 includes the insertion portion 11, the operation portion 12, and the endoscope conduit 13 provided in at least the insertion portion. The suction device 8 can be provided outside the endoscope 1. The endoscope 1 can be connected to the endoscope connection port 2c of the conduit switching device 2 with the endoscope conduit in communication with common conduit 2b3. The endoscope 1 can be a single-use endoscope. The single-use endoscope is disposed after being used once. The single-use endoscope is put in a sterilized package after being manufactured and is subjected to a sterilization treatment in a state of being sealed in the sterilized package, and is shipped.

The operation portion 12 is a part that is provided at the proximal end side of the insertion portion 11. The operation portion 12 performs various operations relating to the endoscope 1.

The operation portion 12 includes a grasping portion 12a, a bending operation lever 12b, and an operation button 12c, for example.

The grasping portion 12a is a part at which an operator grasps the endoscope 1 with a palm.

The bending operation lever 12b is an operation device provided for performing an operation of bending the bending portion 11b with a thumb, for example, of the hand grasping the grasping portion 12a.

The operation button 12c includes a button switch or the like provided for performing an operation relating to image pickup (a release operation or the like), for example.

The conduit selection device 2 is attached to the proximal-end-side opening 13b of the endoscope conduit 13.

The liquid feeding tube 3 and the suction tube 4 are connected to the conduit selection device 2.

The liquid feeding tube 3 is connected to the liquid feeding source 5.

The liquid feeding source 5 is a drip bag or a drip bottle, for example.

A liquid, such as saline, is stored in the liquid feeding source 5.

The liquid feeding source 5 is hung on the caster 6, for example, and is disposed at a position higher than the subject in a weight direction.

Accordingly, the liquid in the liquid feeding source 5 is supplied to the liquid feeding tube 3 due to natural dripping caused by gravity.

The suction tube 4 is connected to the suction device 8 (suction source) via the waste liquid container 7, the suction device 8 being provided outside the endoscope 1.

In a configuration example shown in FIG. 1, the suction device 8 is suction equipment 8a installed on a wall surface of a medical facility, for example.

When the suction tube 4 is in a state of being connected to the suction device 8, suction is performed by the suction device 8, for example.

The suction device 8 suctions fluids (liquids, gasses, and the like) from the subject together with stones, mucus, and the like.

The waste liquid container 7 stores the liquid, the stones, the mucus, and the like in the fluid that is suctioned by the suction device 8.

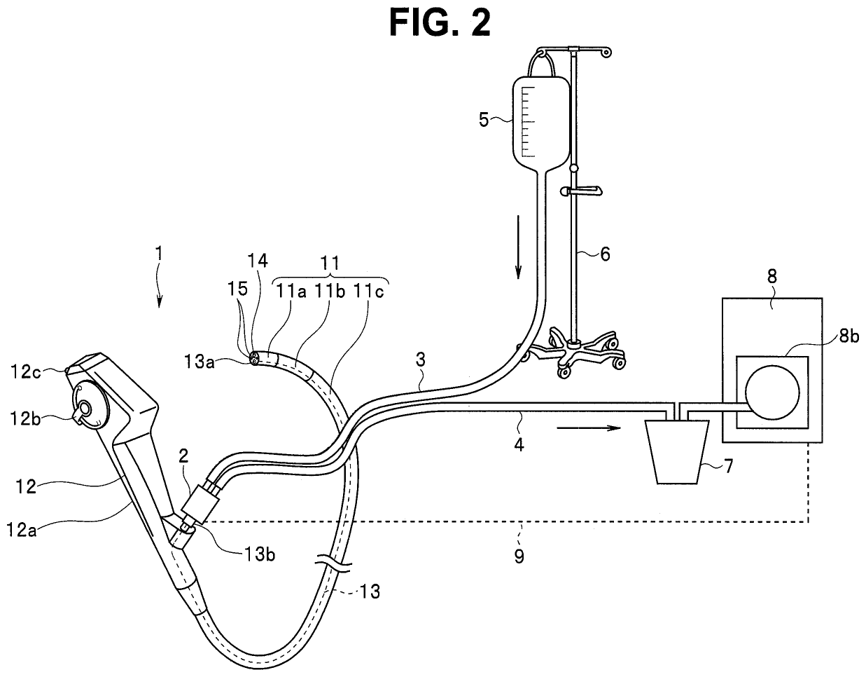

FIG. 2 is a diagram showing another configuration example of the endoscope apparatus of the first embodiment.

Although the suction equipment provided in the medical facility is used as the suction device 8 in FIG. 1, an independent suction pump 8b is used as the suction device 8 in FIG. 2.

The suction device 8 is connected to the conduit selection device 2 via a signal line 9.

As will be described later, the suction device 8 actuates or stops suction in response to a signal transmitted from the conduit selection device 2.

Other configurations in FIG. 2 are substantially the same as the corresponding configurations in FIG. 1.

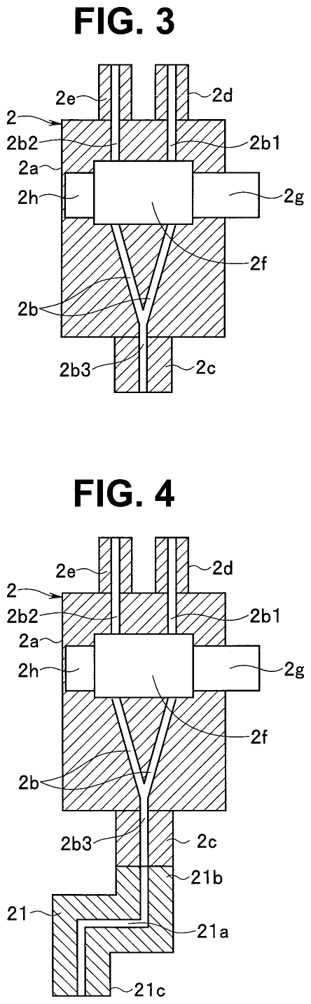

FIG. 3 is a diagram showing a configuration example of the conduit selection device 2 of the first embodiment.

The conduit selection device 2 includes a body 2a, branch conduits 2b, a connection port 2c (endoscope connection port), a fluid supply port 2d (liquid feeding port), a fluid outflow port 2e (suction port, fluid suction port), a selection mechanism 2f (switching mechanism) including an operation device 2g (input controller), and a pressure regulating mechanism 2h.

The body 2a of the conduit selection device 2 houses at least portions of the branch conduits 2b.

The connection port 2c is provided at one end side of the body 2a, and the fluid supply port 2d and the fluid outflow port 2e are provided at the other end side of the body 2a.

The fluid supply port 2d and the fluid outflow port 2e are provided such that extending directions of the fluid supply port 2d and the fluid outflow port 2e from the body 2a (accordingly, directions of conduits in the respective ports 2d, 2e) are parallel to each other, the respective ports 2d, 2e extends toward a merged conduit 2b3 (common conduit) or the respective port 2d extends along the respective port 2e, for example. One or more of the branch conduits 2b and the merged conduit 2b3 can be the common conduit.

The conduit switching device 2 includes the body 2a, an endoscope connection port 2c, the fluid supply port 2d, a fluid suction port 2e, a first conduit 2b1 in the body 2a and the fluid supply port 2d, a second conduit 2b2 in the body and the fluid suction port, the common conduit 2b3 in the body and the endoscope connection port 2c, and a switching mechanism 2f configured to be switched between a first state and a second state. In the first state, the first conduit 2b1 communicates with the common conduit 2b3, and the second conduit 2b2 does not communicate with the common conduit 2b3. In the second state, the first conduit 2b1 does not communicate with the common conduit 2b3, and the second conduit 2b2 communicates with the common conduit 2b3. In the first state, the second conduit 2b2 is at a first pressure, in the second state, the second conduit 2b2 is at a second pressure, and the first pressure is less than the second pressure. In the first state, the second conduit 2b2 suctions from an outside of the body 2a.

The connection port 2c is detachably mounted on the endoscope 1, and communicates with the proximal-end-side opening 13b, which is an end portion of the endoscope conduit 13.

The conduit selection device 2 is positioned by the connection port 2c at a position outside the endoscope 1 and adjacent to the operation portion 12.

The proximal-end-side opening 13b is a female luer port, for example, and the connection port 2c is a male luer port, for example.

By using a luer lock (or a luer taper, a luer fitting or the like) as described above, the connection port 2c is detachably mounted on the port forming the proximal-end-side opening 13b of the endoscope 1.

The branch conduits 2b are in the form of a manifold.

A merged conduit 2b3 is provided at one end side of the branch conduits 2b, and the other end side of the merged conduit 2b3 branches into a first conduit 2b1 and a second conduit 2b2.

The connection port 2c communicates with the merged conduit 2b3.

The fluid supply port 2d communicates with the first conduit 2b1.

The liquid feeding tube 3 is connected to the fluid supply port 2d.

The fluid supply port 2d allows a fluid flowing into the conduit selection device 2 from the outside via the liquid feeding tube 3 to flow to the endoscope conduit 13 via the first conduit 2b1, the merged conduit 2b3, and the connection port 2c.

Accordingly, the first conduit 2b1 and the liquid feeding tube 3 serve as liquid feeding conduits.

The fluid outflow port 2e communicates with the second conduit 2b2.

The suction tube 4 is connected to the fluid outflow port 2e.

The fluid outflow port 2e allows a fluid flowing out from the endoscope conduit 13 and flowing into the conduit selection device 2 to flow to the suction device 8 through the suction tube 4.

The fluid flowing out from the endoscope conduit 13 passes through the connection port 2c, the merged conduit 2b3, and the second conduit 2b2 in the conduit selection device 2.

Accordingly, the second conduit 2b2 and the suction tube 4 serve as suction conduits.

The selection mechanism 2f is attached to the branch conduit 2b.

The selection mechanism 2f selects whether the connection port 2c is enabled to communicate with either the fluid supply port 2d or the fluid outflow port 2e.

Specifically, the selection mechanism 2f selects one from a plurality of states including a first state and a second state.

Accordingly, the selection mechanism 2f may be configured to be capable of further selecting a state other than the first state and the second state.

In the first state, communication is established between the connection port 2c and the fluid supply port 2d, but no communication is established between the connection port 2c and the fluid outflow port 2e.

In the first state, liquid feeding is performed (liquid feeding: on), but suction is not performed (suction: off).

In the second state, no communication is established between the connection port 2c and the fluid supply port 2d, but communication is established between the connection port 2c and the fluid outflow port 2e.

In the second state, suction is performed (suction: on), but liquid feeding is not performed (liquid feeding: off).

According to a selection made by the selection mechanism 2f, the merged conduit 2b3 and the endoscope conduit 13 serve as liquid feeding conduits (a case in which liquid feeding is on) or as suction conduits (a case in which suction is on).

The operation device 2g is operated close to hand by the operator grasping the endoscope 1.

By operating the operation device 2g, selection of the selection mechanism 2f from the plurality of states including the first state and the second state is set.

The pressure regulating mechanism 2h is provided in such a way as to be capable of communicating with the second conduit 2b2.

The pressure regulating mechanism 2h is a suction-conduit-load avoiding device that, when a state in which no communication is established between the connection port 2c and the fluid outflow port 2e (including the first state) is selected by the selection mechanism 2f, reduces a negative pressure in the suction conduits (including the second conduit 2b2) caused by the suction device 8.

Due to such a reduction in negative pressure, it is also possible to reduce a load applied to the suction tube 4, the waste liquid container 7, and the suction device 8.

As shown in FIG. 1 and FIG. 2, a fluid to be fed through the first conduit 2b1 may be supplied from the liquid feeding source 5 to the liquid feeding tube 3 due to natural dripping caused by gravity.

Accordingly, even when a state in which no communication is established between the connection port 2c and the fluid supply port 2d (including the second state) is selected by the selection mechanism 2f, there is no possibility of a large load being applied to the first conduit 2b1 (and the liquid feeding tube 3 and the liquid feeding source 5).

Therefore, a device that reduces a load on the first conduit 2b1 may not be provided.

According to the first embodiment having such a configuration, the conduit selection device 2 is directly attached to the port forming the proximal-end-side opening 13b formed at the operation portion 12 of the endoscope 1 via the connection port 2c and hence, the operator can easily perform an operation of switching, close to hand, between the liquid feeding conduit and the suction conduit at a timing.

When a rotation angle is adjusted in mounting the connection port 2c on the endoscope 1, the operator can perform switching with greater ease of operation.

When an external conduit selection device 2 is attached to a first endoscope having a liquid feeding function but not having a suction function, the first endoscope can be used as a second endoscope having a liquid feeding function and a suction function.

Further, the conduit selection device 2 is detachably mounted on the endoscope 1 and hence, if the conduit selection device 2 is not attached to the endoscope 1, the endoscope 1 can be directly used as a first endoscope.

Accordingly, for both a manufacturer and a medical institution, it may become unnecessary to prepare two products, that is, the first endoscope and the second endoscope.

Therefore, the medical institution can lower an introduction cost, and the manufacturer can also lower manufacturing costs due to simplification of manufacturing lines.

In a conventional endoscope that can perform liquid feeding and suction, an endoscope conduit branches in an operation portion. However, in the present embodiment, a branch to the endoscope conduit 13 may not be provided.

Therefore, in cleaning the endoscope conduit 13, it may become unnecessary to clean a conduit that is not used and only minimum cleaning may be required and hence, cleaning work can be reduced, thus allowing the medical institution to lower a management cost.

A three-way cock is known as a device that can switch conduits. However, a tube for a liquid feeding conduit and a tube for a suction conduit separately extend in different two directions in the three-way cock and hence, the three-way cock reduces ease of operation of an endoscope.

In contrast, according to the first embodiment, the fluid supply port 2d and the fluid outflow port 2e are provided parallel to each other and hence, there is an advantage that the liquid feeding tube 3 and the suction tube 4 extend in the same direction, thus preventing complicated routing.

In a case in which the three-way cock is adopted, a load is applied to the suction conduit if a suction device is being driven at the time of switching the conduit to the liquid feeding conduit.

In addition to the above, at the time of switching the conduit, the three-way cock brings a state in which all conduits are connected to each other.

In contrast, according to the first embodiment, the pressure regulating mechanism 2h is provided in the conduit selection device 2 and hence, even when the suction device 8 is actuated in a state in which no communication is established between the connection port 2c and the fluid outflow port 2e (including the first state), it is possible to reduce a load applied to the suction conduit also at the time of switching the conduits.

As a consequence, with the conduit selection device 2 or the endoscope apparatus including the conduit selection device 2, the operator grasping the endoscope 1 can switch the conduits close to hand with excellent ease of operation and hence, an endoscope including the endoscope conduit 13 with a branch may not be prepared, thus reducing costs.

### Second Embodiment

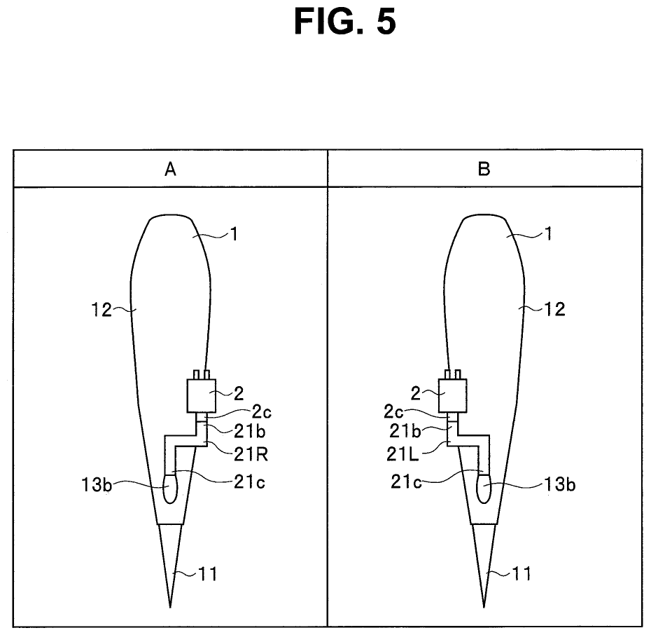

FIG. 4 and FIG. 5 show a second embodiment of the present disclosure.

FIG. 4 is a diagram showing an example in which a joint part is used in mounting the conduit selection device 2 of the second embodiment on the endoscope 1.

In the second embodiment, components substantially the same as the corresponding components in the first embodiment are given the same reference symbols and the description of such components will be omitted when appropriate, and points that make the second embodiment different from the first embodiment will be mainly described.

A joint part 21 is a pipe that includes a conduit 21a in the inside and that is bent into a hook shape (substantially Z shape or curved shape), for example.

A connection port 21c is provided at one end side of the joint part 21 and a connection port 21b is provided at the other end side of the joint part 21, the connection port 21c being connected to the port forming the proximal-end-side opening 13b of the endoscope 1, the connection port 21b being connected to the connection port 2c of the conduit selection device 2.

FIG. 5 is a table showing examples of the joint part 21 that corresponds to a dominant hand of the operator in the second embodiment.

Column A in FIG. 5 shows an example in which the conduit selection device 2 is mounted on the endoscope 1 with a right handed joint part 21R.

By using the joint part 21R bent into a hook shape as shown in the drawing, an axis of the conduit selection device 2 is shifted toward a right hand being the dominant hand and hence, the operator can more easily perform a right-hand operation.

Column B in FIG. 5 shows an example in which the conduit selection device 2 is mounted on the endoscope 1 with a left handed joint part 21L.

By using the joint part 21L bent into a hook shape as shown in the drawing, an axis of the conduit selection device 2 is shifted toward a left hand being the dominant hand and hence, the operator can more easily perform a left-hand operation.

The same configuration of the operation device 2g is adopted for the right handed joint part 21R and the left handed joint part 21L.

According to the second embodiment having such a configuration, it is possible to obtain advantageous effects substantially the same as the advantageous effects of the above-described first embodiment.

In addition to the above, the operator can more easily perform an operation corresponding to the dominant hand of the operator.

### Third Embodiment

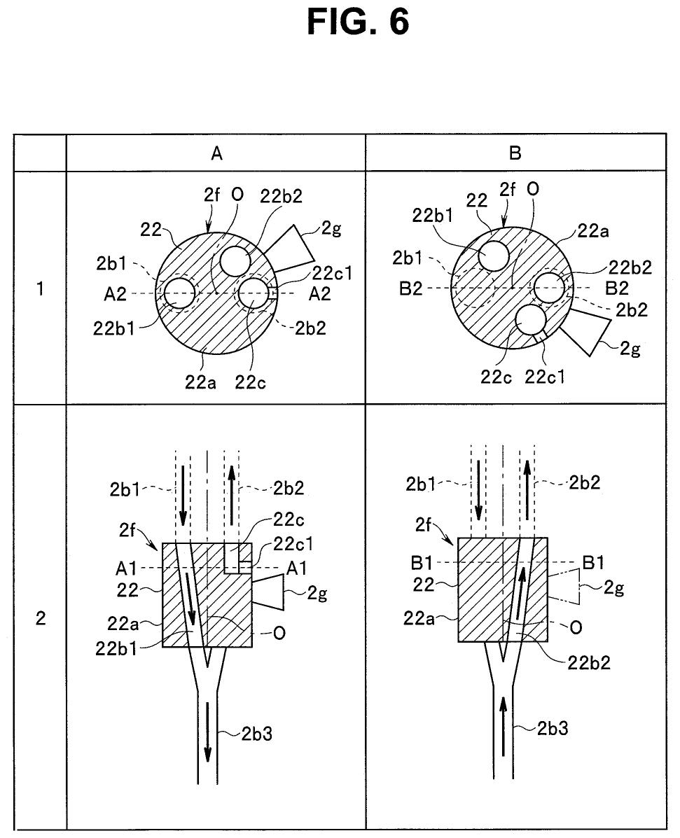

FIG. 6 shows a third embodiment of the present disclosure.

FIG. 6 is a table showing a configuration example of a selection mechanism 2f of a conduit selection device 2 of the third embodiment.

In the third embodiment, components substantially the same as the corresponding components in the first and second embodiments are given the same reference symbols and the description of such components will be omitted when appropriate, and points that make the third embodiment different from the first and second embodiments will be mainly described.

In FIG. 6, column 1A shows a cross-sectional view taken along line A1-A1 in column 2A, column 2A shows a cross-sectional view taken along line A2-A2 in column 1A, column 1B shows a cross-sectional view taken along line B1-B1 in column 2B, and column 2B shows a cross-sectional view taken along line B2-B2 in column 1B.

The selection mechanism 2f includes a rotor 22 that rotates with respect to the body 2a of the conduit selection device 2 about an axis of rotation O.

The selection mechanism 2f selects one from the plurality of states including the first state and the second state according to a rotation angle of the rotor 22.

Examples of the plurality of states other than the first state and the second state include a state in the course of switching between the first state and the second state (the same applies hereinafter).

The operation device 2g formed of a handle or the like is integrally provided on the rotor 22. When the operation device 2g is operated, the rotor 22 rotates about the axis of rotation O.

The rotor 22 includes conduits 22b1, 22b2 and an air suction conduit 22c in a rotor body 22a having a columnar shape, the conduits 22b1, 22b2 forming portions of the branch conduits 2b.

The conduit 22b1, the conduit 22b2, and the air suction conduit 22c are disposed in the rotor body 22a at different angular positions about the axis of rotation O.

Columns 1A, 2A in FIG. 6 show the first state.

The first conduit 2b1 communicates with the merged conduit 2b3 via the conduit 22b1, but the second conduit 2b2 does not communicate with the merged conduit 2b3.

Therefore, the liquid feeding source 5 is connected to the endoscope conduit 13, so that a liquid is fed to the endoscope conduit 13.

In the first state, the second conduit 2b2 communicates with the air suction conduit 22c, and the air suction conduit 22c communicates with an external atmosphere via a leakage prevention stopper 22c1 (sealing member) formed of a check valve or the like.

Accordingly, the suction device 8 suctions air from the outside and hence, a load on the suction conduit is reduced.

As a consequence, the pressure regulating mechanism 2h in the present embodiment is a mechanism that, when the first state is selected by the selection mechanism 2f, switches the second conduit 2b2 such that communication is established between the fluid outflow port 2e and the external atmosphere.

Specifically, the pressure regulating mechanism 2h includes the air suction conduit 22c equipped with the leakage prevention stopper 22c1.

The leakage prevention stopper 22c1 prevents a liquid or the like remaining in the suction conduit from leaking to the outside via the air suction conduit 22c when the suction device 8 stops unintentionally. A part of the second conduit 2b2 has the sealing member 22c1 to prevent a fluid inside of the second conduit 2b2 from leaking to outside of the second conduit 2b2.

In the present embodiment, the first state is a state in which communication is established between the connection port 2c and the fluid supply port 2d, no communication is established between the connection port 2c and the fluid outflow port 2e, and communication is established between the fluid outflow port 2e and the external atmosphere.

Columns 1B, 2B in FIG. 6 show the second state.

The first conduit 2b1 does not communicate with the merged conduit 2b3, but the second conduit 2b2 communicates with the merged conduit 2b3 via the conduit 22b2.

In this state, the second conduit 2b2 does not communicate with the air suction conduit 22c.

Therefore, the suction device 8 is connected to the endoscope conduit 13, so that suction is performed through the endoscope conduit 13.

The liquid feeding source 5 does not communicate with the endoscope conduit 13, so that liquid feeding is stopped.

According to the third embodiment having such a configuration, it is possible to obtain advantageous effects substantially the same as the advantageous effects of the above-described first and second embodiment.

In addition to the above, it is possible to switch the conduits by performing a rotation operation of the rotor 22 with the operation device 2g.

### Fourth Embodiment

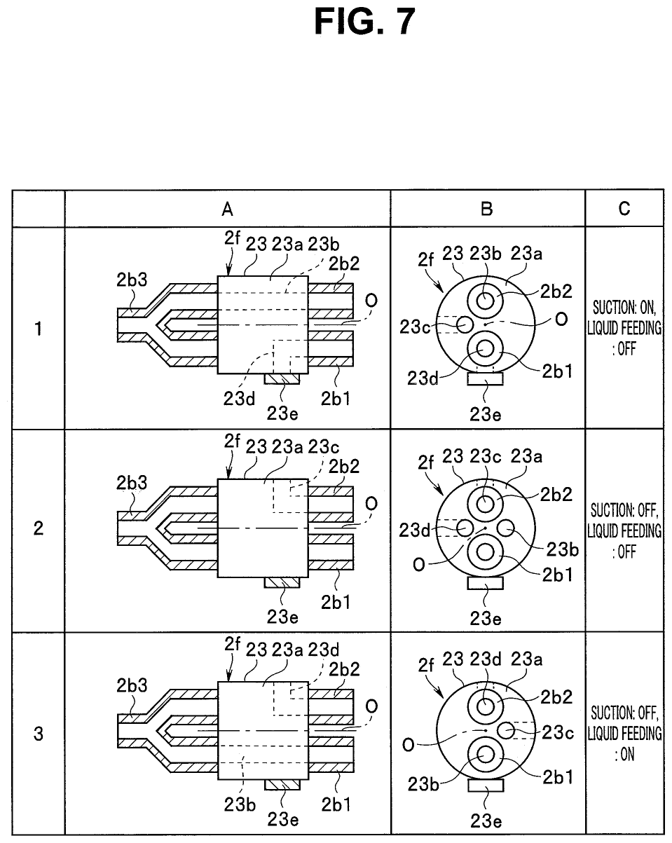

FIG. 7 shows a fourth embodiment of the present disclosure.

FIG. 7 is a table showing a configuration example of a selection mechanism 2f of the conduit selection device 2 of the fourth embodiment.

In the fourth embodiment, components substantially the same as the corresponding components in the first to third embodiments are given the same reference symbols and the description of such components will be omitted when appropriate, and points that make the fourth embodiment different from the first to third embodiments will be mainly described.

The selection mechanism 2f includes a rotor 23 that rotates with respect to the body 2a of the conduit selection device 2 about the axis of rotation O.

The selection mechanism 2f selects one from the plurality of states including the above-described first state, the above-described second state, and a third state according to a rotation angle of the rotor 23.

The third state is a state in which no communication is established between the connection port 2c and the fluid supply port 2d and no communication is established between the connection port 2c and the fluid outflow port 2e, but communication is established between the fluid outflow port 2e and the external atmosphere.

The rotor 23 includes a conduit 23b, a first L-shaped conduit 23c, and a second L-shaped conduit 23d in a rotor body 23a having a columnar shape, the conduit 23b forming a portion of the branch conduit 2b.

The conduit 23b penetrates through the rotor body 23a in a direction of the axis of rotation O.

Each of the first L-shaped conduit 23c and the second L-shaped conduit 23d is a conduit that is provided to extend in the direction of the axis of rotation O and, thereafter, is bent to form an L shape, and is open on a peripheral surface of the rotor body 23a.

An opening of the first L-shaped conduit 23c and an opening of the second L-shaped conduit 23d formed on the peripheral surface basically communicate with the external atmosphere.

A peripheral surface of the rotor 23 is provided with a stopper 23e that is fixed (that is, that does not rotate) to the body 2a of the conduit selection device 2.

When the opening of the first L-shaped conduit 23c or the opening of the second L-shaped conduit 23d formed on the peripheral surface is brought into contact with the stopper 23e, the opening is closed, thus being cut off from the external atmosphere.

The conduit 23b, the first L-shaped conduit 23c, and the second L-shaped conduit 23d are disposed in the rotor body 23a at different angular positions about the axis of rotation O.

Specifically, assuming an angular position of the conduit 23b about the axis of rotation O (in a left rotation direction: a counterclockwise) as viewed in a direction shown in a column B in FIG. 7 as 0 degrees, for example, the first L-shaped conduit 23c is at an angular position of 90 degrees, and the second L-shaped conduit 23d is at an angular position of 180 degrees.

Row 1 (respective columns 1A, 1B, 1C, the same applies hereinafter) in FIG. 7 shows the second state.

The first conduit 2b1 communicates with the second L-shaped conduit 23d and, thereafter, is closed by the stopper 23e, and does not communicate with the merged conduit 2b3.

The second conduit 2b2 communicates with the merged conduit 2b3 via the conduit 23b.

Therefore, the suction device 8 is connected to the endoscope conduit 13, so that suction is performed through the endoscope conduit 13 (suction: on).

The liquid feeding source 5 does not communicate with the endoscope conduit 13, so that liquid feeding is stopped by the stopper 23e (liquid feeding: off).

When the rotor 23 is rotated in a right rotation (clockwise) direction by 90 degrees from the state shown in column 1B in FIG. 7, the rotor 23 is brought into a state shown in column 2B in FIG. 7.

When the rotor 23 is rotated in the left rotation (counterclockwise) direction by 90 degrees from the state shown in column 2B in FIG. 7, the rotor 23 is brought into the state shown in column 1B in FIG. 7.

Row 2 in FIG. 7 shows the third state in which the first conduit 2b1 abuts against an end surface of the rotor body 23a in the direction of the axis of rotation O, thus being closed, and does not communicate with the merged conduit 2b3.

The second conduit 2b2 communicates with the external atmosphere via the first L-shaped conduit 23c, and does not communicate with the merged conduit 2b3.

Therefore, neither the suction device 8 nor the liquid feeding source 5 communicates with the endoscope conduit 13, and suction and liquid feeding are stopped (suction: off, liquid feeding: off).

In such a state, the suction device 8 suctions air from the outside via the first L-shaped conduit 23c, so that a load on the suction conduit is reduced.

The switching mechanism 2f is configured to be switched between the first state (row 1), the second state (row 3), and the third state (row 2). In the third state, the first conduit 2b1 is prevented from communicating with the common conduit 2b3. In the third state, the second conduit 2b2 is at a third pressure, the third pressure is less than the second pressure of the second conduit 2b2 in the second state. In the third state, the first conduit 2b1 cannot be in communication with the common conduit, the second conduit 2b2 cannot be in communication with the common conduit, and the second conduit 2b2 can suction from outside of the body 2a.

When the rotor 23 is rotated in the right rotation (clockwise) direction by 90 degrees from the state shown in column 2B in FIG. 7, the rotor 23 is brought into a state shown in column 3B in FIG. 7.

When the rotor 23 is rotated in the left rotation (counterclockwise) direction by degrees from the state shown in column 3B in FIG. 7, the rotor 23 is brought into the state shown in column 2B in FIG. 7.

Row 3 in FIG. 7 shows the first state in which the first conduit 2b1 communicates with the merged conduit 2b3 via the conduit 23b.

The second conduit 2b2 communicates with the external atmosphere via the second L-shaped conduit 23d, and does not communicate with the merged conduit 2b3.

Therefore, the suction device 8 does not communicate with the endoscope conduit 13, so that suction is stopped (suction: off).

The liquid feeding source 5 is connected to the endoscope conduit 13, so that a liquid is fed to the endoscope conduit 13 (liquid feeding: on).

In such a state, the suction device 8 suctions air from the outside via the second L-shaped conduit 23d, so that a load on the suction conduit is reduced.

Although not shown in the drawing, in the same manner as the third embodiment, the operation device 2g formed of the handle or the like may be integrally provided on the rotor 23, or a leakage prevention stopper formed of a check valve or the like may be provided in each of the first L-shaped conduit 23c and the second L-shaped conduit 23d.

According to the fourth embodiment having such a configuration, it is possible to obtain advantageous effects substantially the same as the advantageous effects of the above-described first to third embodiments.

In addition to the above, the selection mechanism 2f can further select the third state in addition to the first state and the second state and hence, it is also possible to handle a procedure that uses neither suction nor liquid feeding.

Further, when the suction device 8 is in actuation in the first state or the third state, it is possible to reduce a load on the suction conduit.

### Fifth Embodiment

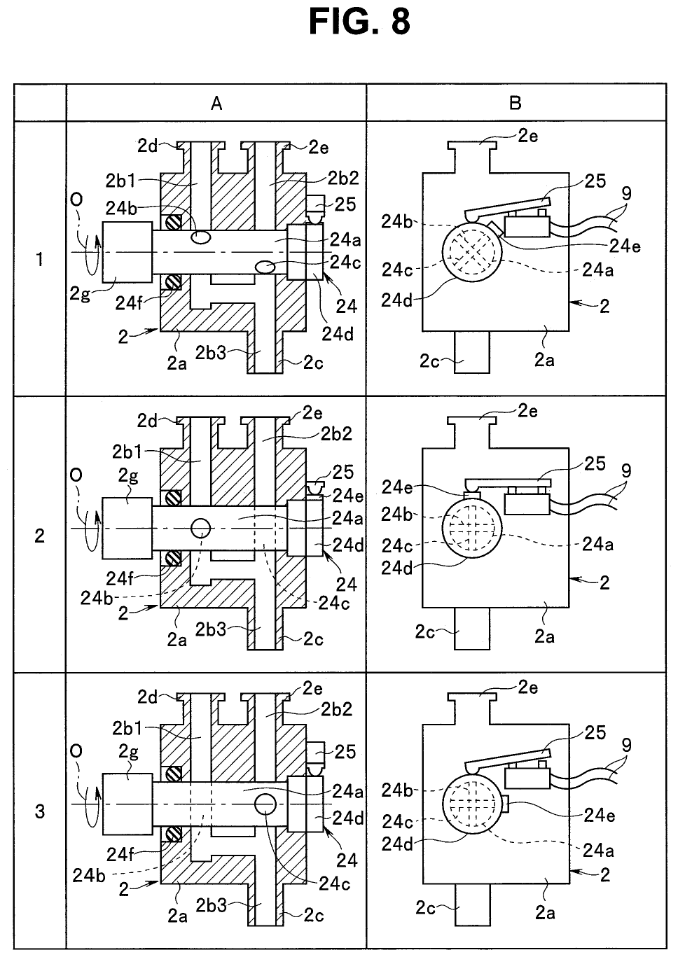

FIG. 8 shows a fifth embodiment of the present disclosure.

FIG. 8 is a table showing a configuration example of a selection mechanism 2f of a conduit selection device 2 of the fifth embodiment.

In the fifth embodiment, components substantially the same as the corresponding components in the first to fourth embodiments are given the same reference symbols and the description of such components will be omitted when appropriate, and points that make the fifth embodiment different from the first to fourth embodiments will be mainly described.

Column B in FIG. 8 shows side views of the conduit selection device 2 shown in column A as viewed from a right side. The switching mechanism 2f is configured to be switched between the first state (row 3), the second state (row 2), and the third state (row 1). In the third state, the first conduit 2b1 is prevented from communicating with the common conduit 2b3. In the third state, the second conduit 2b2 is at a third pressure, the third pressure is less than the second pressure of the second conduit 2b2 in the second state. In the third state, the first conduit 2b1 cannot be in communication with the common conduit, the second conduit 2b2 cannot be in communication with the common conduit, and the second conduit 2b2 can suction from outside of the body 2a.

In the present embodiment, each of the fluid supply port 2d and the fluid outflow port 2e is a luer lock port, for example (however, needless to say, not limited to the luer lock port), the fluid supply port 2d communicating with the first conduit 2b1, the fluid outflow port 2e communicating with the second conduit 2b2.

The selection mechanism 2f includes a rotor 24 that rotates with respect to the body 2a of the conduit selection device 2 about the axis of rotation O.

The selection mechanism 2f selects one from the plurality of states including the first state, the second state, and the third state according to a rotation angle of the rotor 24.

The rotor 24 includes a conduit 24b and a conduit 24c in a rotor body 24a having a columnar shape, the conduit 24b and the conduit 24c forming portions of the branch conduits 2b.

In the direction of the axis of rotation O, the conduit 24b is provided at a position corresponding to the first conduit 2b1, and the conduit 24c is provided at a position corresponding to the second conduit 2b2.

The conduit 24b and the conduit 24c penetrate through the rotor body 24a in a direction (a radial direction) orthogonal to the axis of rotation O at different angular positions about the axis of rotation O.

Specifically, a center axis of the conduit 24b is orthogonal to the axis of rotation O, and a center axis of the conduit 24c is orthogonal to the axis of rotation O.

Although the center axis of the conduit 24b and the center axis of the conduit 24c have a twisted positional relationship, a direction of the center axis of the conduit 24b is orthogonal to a direction of the center axis of the conduit 24c.

A flange 24d is provided at an end portion of the rotor body 24a on one end side, and a portion of the flange 24d in the direction of the axis of rotation O protrudes to the outside of the body 2a of the conduit selection device 2.

A protruding portion 24e that protrudes outward in the radial direction is provided on an outer periphery of the portion of the flange 24d that protrudes to the outside of the body 2a.

A switch 25 (sensor) formed of a microswitch or the like (or a sensor) is attached to an outer surface of the body 2a on a side close to the flange 24d.

For example, the switch 25 is configured such that the switch 25 is off when the switch 25 is brought into contact with the outer periphery of the flange 24d other than the protruding portion 24e, and the switch 25 is on when the switch 25 is brought into contact with the protruding portion 24e.

Specifically, the protruding portion 24e is provided, in a circumferential direction about the axis of rotation O, at a position that causes the switch 25 to be on when the second conduit 2b2 communicates with the merged conduit 2b3 via the conduit 24c (that is, when suction is on), and that causes the switch 25 to be off when the second conduit 2b2 does not communicate with the merged conduit 2b3 via the conduit 24c.

The switch 25 forming the pressure regulating mechanism 2h is connected to a control circuit of the suction pump 8b via the signal line 9 (see FIG. 2).

When the second state is selected by the selection mechanism 2f (when the rotor 24 assumes a rotation angle at which the second state is selected), so that the switch 25 is turned on, the control circuit actuates the suction pump 8b to perform suction.

When a state other than the second state (including the first state and the third state) is selected by the selection mechanism 2f (when the rotor 24 assumes an angle other than the rotation angle at which the second state is selected), so that the switch 25 is turned off, the control circuit stops the suction pump 8b to prevent suction.

With such a configuration, suction is not performed by the suction pump 8b except when suction is on and hence, it is possible to reduce a load on the suction conduit.

The sensor 25 is configured to detect one of the first state and the second state of the switching mechanism 2f, generate a first signal when the first state is detected, and generate a second signal when the second state is detected. The first signal can actuate or turn on the suction device 8, and the second signal can prevent actuation of the suction device 8 or turn off the suction device 8. The conduit switching device 2f can further comprise a processor. The sensor can be configured to detect one of the first state and the second state, generate the first signal when the first state is detected, and generate the second signal when the second state is detected. The processor can be configured to receive one of the first signal or the second signal from the sensor, and actuate the suction device to perform suction when the first signal is received and stop the suction device to prevent suction when the second signal is received. The rotor 24 rotates with respect to the body 2a about a longitudinal axis of the body 2a, and the sensor 25 generates one of the first signal and the second signal in response to a rotation angle of the rotor 24. In the first state, the rotation angle is within a first angle range, and in the second state, the rotation angle is within a second angle range. The second angle range can be smaller than the first angle range. The angle range can be measured by the protruding portion 24e and the sensor 25, or a rotary encoder.

An O-shaped ring 24f (sealing member) is provided on an outer periphery of the rotor body 24a on the other end side at a position between the rotor body 24a and the body 2a. The O-shaped ring 24f serves as a seal that prevents leakage of a liquid.

The operation device 2g formed of a dial or the like is integrally provided on the rotor 24 at the other end of the rotor body 24a.

When the operator rotates the operation device 2g about the axis of rotation O, the rotor 24 also integrally rotates with the operation device 2g.

A surface of the operation device 2g can be formed of a dial or the like, have a surface shape formed by knurls or the like, or a material such as rubber be provided on the surface of the operation device 2g so as to allow the operation device 2g to be easily operated.

Row 1 in FIG. 8 shows the third state (liquid feeding: off, suction: off).

In the third state, no communication is established between the connection port 2c and the fluid supply port 2d, and no communication is established between the connection port 2c and the fluid outflow port 2e.

Row 2 in FIG. 8 shows the second state obtained by rotating the rotor 24 by 45 degrees in the left rotation direction from the state shown in row 1 as viewed in a direction shown in column B (liquid feeding: off, suction: on).

In the second state, no communication is established between the connection port 2c and the fluid supply port 2d, but communication is established between the connection port 2c and the fluid outflow port 2e via the conduit 24c.

In the second state, an opening of the connection port 2c, the merged conduit 2b3, the conduit 24c, the second conduit 2b2, and an opening of the fluid outflow port 2e form a linear conduit as shown in column 2A.

Such a configuration allows a treatment instrument, a guide wire, or the like to be easily inserted through the endoscope conduit 13 via the conduit selection device 2.

In contrast, the first conduit 2b1 is bent into an L shape, and merges with the merged conduit 2b3.

Row 3 in FIG. 8 shows the first state obtained by rotating the rotor 24 by 45 degrees in the right rotation direction from the state shown in row 1 as viewed in the direction shown in column B (liquid feeding: on, suction: off).

In the first state, communication is established between the connection port 2c and the fluid supply port 2d via the conduit 24b, but no communication is established between the connection port 2c and the fluid outflow port 2e.

According to the fifth embodiment having such a configuration, it is possible to obtain advantageous effects substantially the same as the advantageous effects of the above-described first to fourth embodiments.

In addition to the above, when the conduit selection device 2 is attached to the endoscope 1, the operation device 2g, which is formed of a dial or the like, is located at a position close to the grasping portion 12a and hence, the operator can easily perform rotation operation of the operation device 2g close to hand.

### Sixth Embodiment

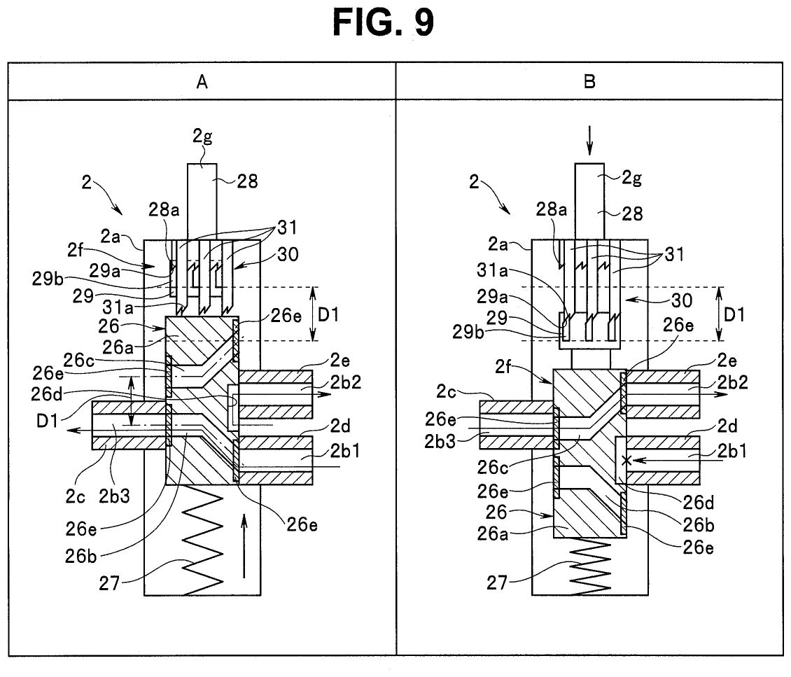

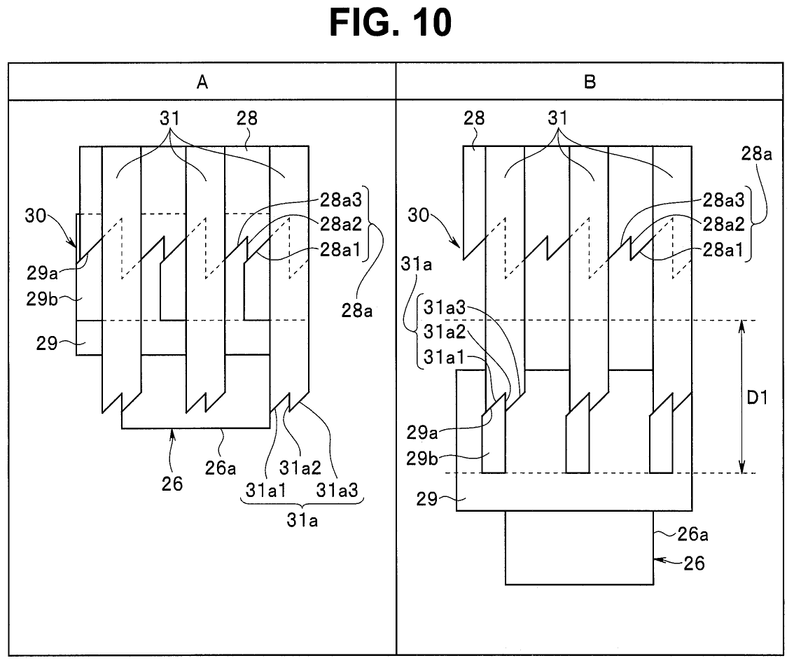

FIG. 9 and FIG. 10 show a sixth embodiment of the present disclosure.

FIG. 9 is a table showing a schematic configuration example of a selection mechanism 2f of a conduit selection device 2 of the sixth embodiment.

FIG. 10 is a table schematically showing a knock cam mechanism 30 of the selection mechanism 2f in the sixth embodiment in an enlarged manner.

In the sixth embodiment, components substantially the same as the corresponding components in the first to fifth embodiments are given the same reference symbols and the description of such components will be omitted when appropriate, and points that make the sixth embodiment different from the first to fifth embodiments will be mainly described.

Columns A in FIG. 9 and FIG. 10 show the first state (liquid feeding: on, suction: off), and columns B in FIG. 9 and FIG. 10 show the second state (liquid feeding: off, suction: on).

The selection mechanism 2f in the present embodiment includes a slide mechanism 26 formed as the knock cam mechanism 30.

FIG. 9 and FIG. 10 schematically show the knock cam mechanism 30 in a state of being developed in the circumferential direction, the knock cam mechanism 30 being formed into a cylindrical shape.

The slide mechanism 26 includes a slide body 26a (valve, porting body) and a button 28 (the operation device 2g), and selects one from the plurality of states including the first state and the second state according to a slide position of the slide body 26a.

The switching mechanism 2f includes the porting body 26a slidably movable relative to the body 2a between a first position and a second position, and an input controller configured to receive an external force and to translate the external force to the porting body 26a to switch the porting body 26a between the first position and the second position. The input controller can be the operation device 2g, button, lever, dial, etc.

The slide body 26a includes a conduit 26b and a conduit 26c that form portions of the branch conduits 2b. In other words, the slide body 26a houses at least the portions of the branch conduits 2b.

Each of the conduit 26b and the conduit 26c is provided in the slide body 26a in a penetrating manner.

A leakage prevention stopper 26e made of a sealing material or the like is provided at each of openings of the conduit 26b and the conduit 26c at both ends.

The slide body 26a also has a recessed portion 26d at a position that can face the first conduit 2b1 or the second conduit 2b2 according to the slide position.

In the first state shown in column A in FIG. 9, the recessed portion 26d communicates with both the second conduit 2b2 and the external atmosphere to allow the suction device 8 to suction air from the outside.

In the second state shown in column B in FIG. 9, the recessed portion 26d closes the first conduit 2b1 to stop liquid feeding.

When the button 28 receives an external force in the downward direction in FIG. 9 and FIG. 10 caused by a pressing operation by the operator, the button 28 slides the slide body 26a in the downward direction with the knock cam mechanism 30.

The knock cam mechanism 30 of the selection mechanism 2f further includes an urging member 27 (biasing element), a rotor 29, and cams.

The urging member 27 includes an elastic component, such as a spring, and urges the slide body 26a toward the button 28.

The switching mechanism 2f includes the biasing element 27 configured to bias the porting body 26a toward the input controller 2g, and the knock cam mechanism 30 between the input controller 2g and the porting body 26a. The knock cam mechanism is configured to translate the external force from the input controller 2g to the porting body 26a to switch the porting body 26a between the first position and the second position.

The rotor 29 is disposed between the slide body 26a and the button 28.

The rotor 29 is movable in a sliding direction integrally with the slide body 26a.

The rotor 29 is rotatable about a center axis that is parallel to the sliding direction.

In contrast, rotation of the slide body 26a is stopped by the body 2a, for example, so that the slide body 26a is not rotated integrally with the rotor 29.

The body 2a has a cylindrical inner surface that faces the rotor 29.

A plurality of ribs 31 that protrude toward the rotor 29 are provided on the cylindrical inner surface of the body 2a in the circumferential direction.

A plurality of ribs 29b protruding outward are provided on a cylindrical outer peripheral surface of the rotor 29 in the circumferential direction.

Spaces each formed between two adjacent ribs 31 of the plurality of ribs 31 are grooves that guide the ribs 29b in the sliding direction.

The ribs 29b enter the grooves each formed between the two adjacent ribs 31, so that the rotor 29 is movable in the sliding direction in a state in which rotation of the rotor 29 about the center axis is restricted.

The cam of the knock cam mechanism 30 is provided on each of the body 2a, the button 28, and the rotor 29.

As shown in FIG. 10, a cam 29a inclined in the sliding direction is provided on an end surface of each rib 29b of the rotor 29 on a side that faces the button 28 (an upper side in FIG. 9 and FIG. 10).

A cam 31a is provided on an end surface of each rib 31 on a side close to the slide body 26a (a lower side in FIG. 9 and FIG. 10).

In the development view shown in FIG. 10, each cam 31a includes a first cam 31a1, a step 31a2, and a second cam 31a3, the first cam 31a1 being inclined in the same direction as the cam 29a, the step 31a2 extending in the sliding direction, the second cam 31a3 being inclined in the same direction as the cam 29a.

Cams 28a are provided on an end surface of the button 28 on a side that faces the rotor 29 (a lower side in FIG. 9 and FIG. 10).

In the development view shown in FIG. 10, each cam 28a includes a first cam 28a1, a step 28a2, and a second cam 28a3, the first cam 28a1 being inclined in the same direction as the cam 29a, the step 28a2 extending in the sliding direction, the second cam 28a3 being inclined in the same direction as the cam 29a.

The knock cam mechanism 30 has a known technique. Each time the button 28 receives an external force (a pressing force in the sliding direction), the cams of the knock cam mechanism 30 change a position of the slide body 26a by a distance D1 shown in FIG. 9 and FIG. 10 via the rotor 29.

With such an operation, the first state shown in columns A in FIG. 9 and FIG. 10 and the second state shown in columns B in FIG. 9 and FIG. 10 are switched.

Specifically, in the first state, communication is established between the connection port 2c and the fluid supply port 2d via the conduit 26b, but no communication is established between the connection port 2c and the fluid outflow port 2e.

The fluid outflow port 2e communicates with the external atmosphere via the recessed portion 26d.

Leakage of a liquid or the like in the conduit 26c is prevented by the leakage prevention stoppers 26e provided at openings of the conduit 26c at both ends.

In the first state, when the operator depresses the button 28 to move the button 28 in the sliding direction, a portion of each first cam 28a1 engages with the cam 29a, and the rotor 29 is pressed in the sliding direction.

The first cams 28a1 and the cams 29a are inclined in the sliding direction and hence, the rotor 29 attempts to rotate about the center axis. However, rotation of the rotor 29 is restricted due to engagement between the ribs 31 and the ribs 29b.

As a consequence, the rotor 29 moves in the sliding direction with movement of the button 28 in the state in which the rotation of the rotor 29 about the center axis is restricted.

When the cams 29a are moved and cross over the cams 31a of the ribs 31, the rotor 29 can rotate about the center axis.

The rotor 29 rotates in one direction about the center axis along the first cams 28a1 of the button 28, the rotor 29 being urged toward the button 28 by the urging member 27 via the slide body 26a.

Then, when the cams 29a reach the step 28a2, the rotation of the rotor 29 is restricted.

Thereafter, when the operator stops pressing the button 28, the slide body 26a, the rotor 29, and the button 28 are slid and moved in the upward direction in FIG. 9 and FIG. 10 by an urging force of the urging member 27.

Then, the cams 29a engage with the first cams 31a1 of the ribs 31, and further rotate in one direction along the first cams 31a1.

When the cams 29a reach the step 31a2, rotation of the rotor 29 and movement of the rotor 29 in the sliding direction are restricted.

Therefore, the rotor 29 and the slide body 26a are held in positions shown in columns B in FIG. 9 and FIG. 10.

In the second state shown in columns B in FIG. 9 and FIG. 10, communication is established between the connection port 2c and the fluid outflow port 2e via the conduit 26c.

The first conduit 2b1 is closed by the recessed portion 26d, so that the fluid supply port 2d does not communicate with the connection port 2c.

Leakage of a liquid or the like in the conduit 26b is prevented by the leakage prevention stoppers 26e provided at openings of the conduit 26b at both ends.

In the second state, when the operator depresses the button 28, the button 28 is slid and moved in the downward direction in FIG. 9 and FIG. 10, and a portion of each second cam 28a3 engages with the cam 29a that reaches the step 31a2 and is stopped.

When the operator further depresses the button 28 against the urging force of the urging member 27, the ribs 29b move in the downward direction along the step 31a2.

Therefore, the rotor 29 and the slide body 26a integrally move in the downward direction.

When the cams 29a cross over the step 31a2 and the second cams 31a3, the cams 29a rotate in one direction along the second cams 28a3.

Then, when the cams 29a reach the step 28a2, the rotation of the rotor 29 in one direction is restricted.

Thereafter, when the operator stops pressing the button 28, the slide body 26a, the rotor 29, and the button 28 are slid and moved in the upward direction in FIG. 9 and FIG. 10 by the urging force of the urging member 27.

Then, the cams 29a engage with the second cams 31a3 of the ribs 31, and further rotate in one direction along the second cams 31a3.

There is nothing that prevents movement of the button 28 in the sliding direction and hence, the button 28 continues moving in the upward direction in FIG. 9 and FIG. 10 without any change, and the cam 28a is separated from the cam 29a.

When the rotor 29 is rotated, so that engagement between the cams 29a and the second cams 31a3 is released, each rib 29b enters a next groove, being a groove formed between the two ribs 31.

Then, the rotor 29 and the slide body 26a are slid and moved in the upward direction in FIG. 9 and FIG. 10 by the urging force of the urging member 27.

Although the portion of each second cam 28a3 is in the groove formed between the two adjacent ribs 31, a width of the portion of each second cam 28a3 in the circumferential direction is smaller than a width of the rib 29b in the circumferential direction.

Accordingly, when the ribs 29b enter the grooves, each formed between the two adjacent ribs 31, and are moved in the upward direction, each cam 29a engages with a portion of each first cam 28a1, but does not engage with the second cam 28a3.

When the cams 29a engage with the first cams 28a1, the cams 29a rotate in one direction along the first cams 28a1 within a range of a width in the circumferential direction of the grooves each formed between the two adjacent ribs 31, and the first state shown in column A in FIG. 9 and FIG. 10 is restored.

According to the sixth embodiment having such a configuration, it is possible to obtain advantageous effects substantially the same as the advantageous effects of the above-described first to fifth embodiments.

In addition to the above, liquid feeding and suction can be alternately switched each time an operation of pressing (knocking) the button 28 is performed one time.

By adopting the knocking operation for switching between liquid feeding and suction, the operation can be easily performed even with one hand.

### Seventh Embodiment

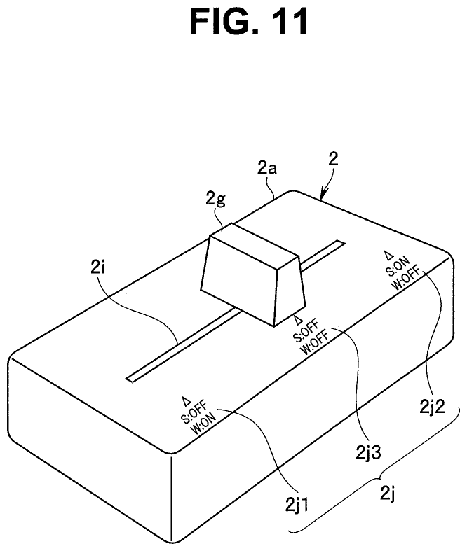

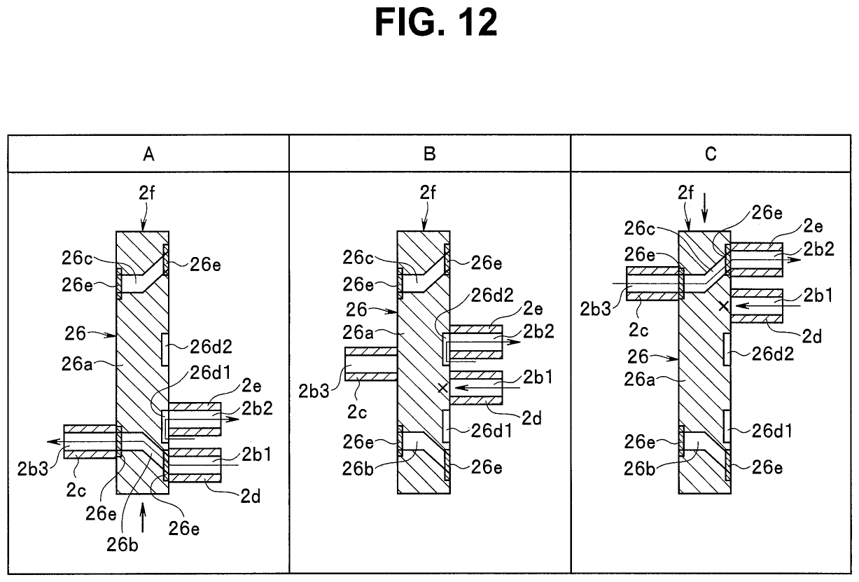

FIG. 11 and FIG. 12 show a seventh embodiment of the present disclosure.

FIG. 11 is a perspective view showing an external appearance of a conduit selection device 2 of the seventh embodiment.

FIG. 12 is a table showing a configuration example of a selection mechanism 2f of the conduit selection device 2 of the seventh embodiment.

In the seventh embodiment, components substantially the same as the corresponding components in the first to sixth embodiments are given the same reference symbols and the description of such components will be omitted when appropriate, and points that make the seventh embodiment different from the first to sixth embodiments will be mainly described.

The selection mechanism 2f in the present embodiment includes a manual slide mechanism 26 (see FIG. 12).

As shown in FIG. 11, the conduit selection device 2 is provided with an operation device 2g configured as a slider that is pinched between fingers.

A linear guide hole 2i is formed in a body 2a of the conduit selection device 2, and the operation device 2g can slide along the guide hole 2i.

The operation device 2g is coupled to the slide body 26a of the slide mechanism 26, which is provided in the conduit selection device 2.

Accordingly, when the operation device 2g is slid, the slide body 26a is also slid and moved integrally with the operation device 2g.

Index marks 2j are provided on a surface of the conduit selection device 2 at a plurality of positions along the guide hole 2i.

The index marks 2j include a first index mark 2j1, a third index mark 2j3, and a second index mark 2j2 in order in a sliding direction of the operation device 2g along the guide hole 2i.

The first index mark 2j1 indicates that when the operation device 2g is moved to a position that matches the first index mark 2j1, the first state is established in which suction (S) is off (OFF) and liquid feeding (W) is on (ON).

The second index mark 2j2 indicates that when the operation device 2g is moved to a position that matches the second index mark 2j2, the second state is established in which suction (S) is on (ON) and liquid feeding (W) is off (OFF).

The third index mark 2j3 indicates that when the operation device 2g is moved to a position that matches the third index mark 2j3, the third state is established in which suction (S) is off (OFF) and liquid feeding (W) is off (OFF).

As described above, the conduit selection device 2 of the present embodiment is configured such that the first state is established by sliding the operation device 2g in one direction from the third state, in which the operation device 2g is located at a center of a slide range, and the second state is established by sliding the operation device 2g in the other direction.

As shown in FIG. 12, the slide mechanism 26 includes the slide body 26a.

In the same manner as the sixth embodiment, the slide body 26a includes the conduit 26b, the conduit 26c, and the leakage prevention stoppers 26e provided at the openings of each of the conduit 26b and the conduit 26c at both ends.

Further, in the slide body 26a, a first recessed portion 26d1 and a second recessed portion 26d2 are provided on a surface of the slide body 26a that faces the fluid supply port 2d and the fluid outflow port 2e.

The first recessed portion 26d1 and the second recessed portion 26d2 are provided at different positions in the sliding direction between the conduit 26b and the conduit 26c.

The first recessed portion 26d1 is provided at a position closer to the conduit 26b than the second recessed portion 26d2.

The second recessed portion 26d2 is provided at a position closer to the conduit 26c than the first recessed portion 26d1.

In the first state shown in column A in FIG. 12, communication is established between the connection port 2c and the fluid supply port 2d via the conduit 26b, but no communication is established between the connection port 2c and the fluid outflow port 2e.

The fluid outflow port 2e communicates with the external atmosphere via the first recessed portion 26d1.

Leakage of a liquid or the like in the conduit 26c is prevented by the leakage prevention stoppers 26e.

In the third state shown in column B in FIG. 12, the first conduit 2b1 is closed by the surface of the slide body 26a at a portion located between the first recessed portion 26d1 and the second recessed portion 26d2, so that the fluid supply port 2d does not communicate with the connection port 2c.

The fluid outflow port 2e does not communicate with the connection port 2c, but communicates with the external atmosphere via the second recessed portion 26d2.

Leakage of a liquid or the like in the conduit 26b and the conduit 26c is prevented by the leakage prevention stoppers 26e.

In the second state shown in column C in FIG. 12, communication is established between the connection port 2c and the fluid outflow port 2e via the conduit 26c.

The first conduit 2b1 is closed by the surface of the slide body 26a at a portion located between the second recessed portion 26d2 and the conduit 26c, so that the fluid supply port 2d does not communicate with the connection port 2c.

Leakage of a liquid or the like in the conduit 26b is prevented by the leakage prevention stoppers 26e. The switching mechanism 2f is configured to be switched between the first state (column A), the second state (column C), and the third state (column B). In the third state, the first conduit 2b1 is prevented from communicating with the common conduit 2b3. In the third state, the second conduit 2b2 is at a third pressure, the third pressure is less than the second pressure of the second conduit 2b2 in the second state. In the third state, the first conduit 2b1 cannot be in communication with the common conduit, the second conduit 2b2 cannot be in communication with the common conduit, and the second conduit 2b2 can suction from outside of the body 2a.

According to the seventh embodiment having such a configuration, it is possible to obtain advantageous effects substantially the same as the advantageous effects of the above-described first to sixth embodiments.

In addition to the above, the selection mechanism 2f can further select the third state in addition to the first state and the second state and hence, it is also possible to handle a procedure that uses neither suction nor liquid feeding.

Further, when the suction device 8 is in actuation in the first state or the third state, it is possible to reduce a load on the suction conduit.

### Eighth Embodiment

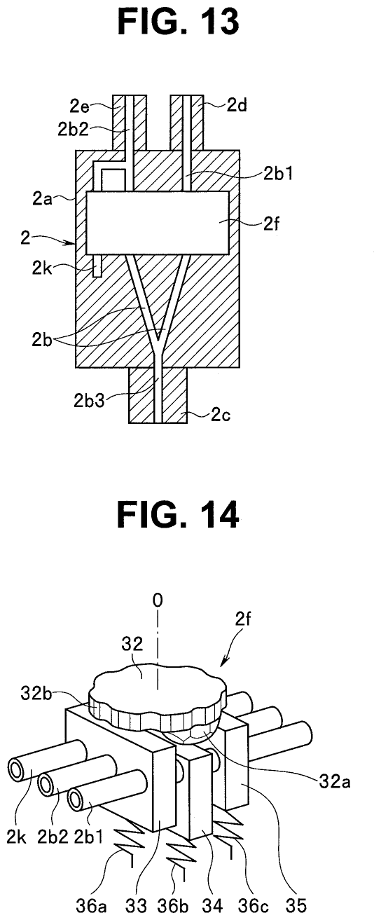

FIG. 13 to FIG. 18 show an eighth embodiment of the present disclosure.

FIG. 13 is a diagram showing a configuration example of a conduit selection device 2 of the eighth embodiment.

In the eighth embodiment, components substantially the same as the corresponding components in the first to seventh embodiments are given the same reference symbols and the description of such components will be omitted when appropriate, and points that make the eighth embodiment different from the first to seventh embodiments will be mainly described.

The conduit selection device 2 of the present embodiment further includes an air suction conduit 2k (third conduit) in addition to the components shown in FIG. 3.

One end of the air suction conduit 2k communicates with the fluid outflow port 2e, and the other end of the air suction conduit 2k communicates with the external atmosphere. As shown in FIGS. 13 to 27, the third conduit 2k can communicate between the second conduit and the outside of the body 2a. In the first state, the switching mechanism 2f is configured to suction fluid from the outside of the body 2a via the third conduit 2k, and prevent the second conduit 2b2 from suctioning fluid from the common conduit 2b3. In the second state, the switching mechanism 2f is configured to suction fluid from the common conduit 2b3 via the second conduit 2b2, and prevent the third conduit 2k from suctioning fluid from the outside of the body 2a.

A selection mechanism 2f also serves as the operation device 2g and the pressure regulating mechanism 2h shown in FIG. 3, and selects the first conduit 2b1, the second conduit 2b2, or the air suction conduit 2k as will be described below.

FIG. 14 is a perspective view showing a configuration example of the selection mechanism 2f of the conduit selection device 2 of the eighth embodiment.

The selection mechanism 2f includes a rotary plate 32, a first conduit switching plate 33 (porting body), a second conduit switching plate 34, a third conduit switching plate 35, and urging members 36a, 36b, 36c.

The rotary plate 32 is a rotor that rotates with respect to the body 2a about the axis of rotation O.

The selection mechanism 2f selects one from the plurality of states including the first state, the second state, and the third state according to a rotation angle of the rotary plate 32.

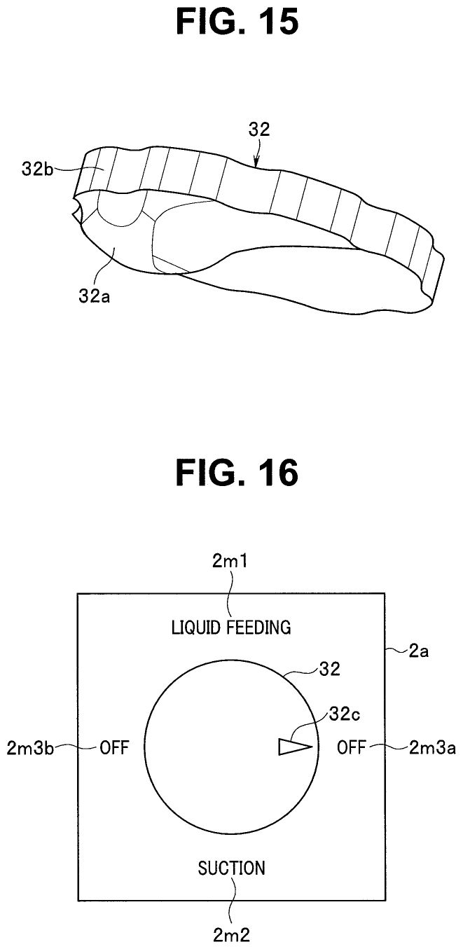

FIG. 15 is a perspective view showing a configuration example of the rotary plate 32 of the selection mechanism 2f of the eighth embodiment as viewed from a cam 32a side.

The rotary plate 32 is a cam-equipped disk in which a cam 32a having unevenness is provided on one surface of the cam-equipped disk in the direction of the axis of rotation O.

The rotary plate 32 is disposed such that a surface of the rotary plate 32 that is provided with the cam 32a faces the first conduit switching plate 33, the second conduit switching plate 34, and the third conduit switching plate 35.

The switching mechanism 2f includes the porting body 33 slidably movable relative to the body 2a between a first position and a second position, and an input controller configured to receive the external force and to translate the external force to the porting body to switch the porting body between the first position and the second position. The input controller can be the rotary plate 32, button, lever, dial, etc. The switching mechanism 2f includes the biasing element 27 configured to bias the porting body 26a toward the input controller 2g, and the knock cam mechanism 30 between the input controller 2g and the porting body 26a. The knock cam mechanism is configured to translate the external force from the input controller 2g to the porting body 26a to switch the porting body 26a between the first position and the second position.

A peripheral surface 32b of the rotary plate 32 having a disk shape may be knurled so as to facilitate a rotation operation of the rotary plate 32.

Alternatively, the rotation operation of the rotary plate 32 may be facilitated by covering the peripheral surface 32b of the rotary plate 32 and a surface of the rotary plate 32 on a side opposite to the cam 32a with a rubber cap or the like that can generate friction.

FIG. 16 is a diagram showing the rotary plate 32 disposed on a surface of the body 2a of the conduit selection device 2 of the eighth embodiment.

The surface of the rotary plate 32 is provided with an index mark 32c indicating one direction in the circumferential direction of a disk shape.

In contrast, a liquid feeding index mark 2m1, an off index mark 2m3a, a suction index mark 2m2, and an off index mark 2m3b are provided on the surface of the body 2a at angular positions around the rotary plate 32 at 90 degree intervals in order in the right rotation (clockwise) direction.

A case in which the index mark 32c points to the liquid feeding index mark 2m1 indicates that liquid feeding is on and suction is off.

A case in which the index mark 32c points to the suction index mark 2m2 indicates that suction is on and liquid feeding is off.

A case in which the index mark 32c points to the off index mark 2m3a or the off index mark 2m3b indicates that both liquid feeding and suction are off.

The first conduit switching plate 33, the second conduit switching plate 34, and the third conduit switching plate 35 are disposed in such a way as to face intermediate portions of the first conduit 2b1, the second conduit 2b2, and the air suction conduit 2k and to be movable in a direction intersecting with the respective conduits (specifically, in a direction orthogonal to and crossing the respective conduits).

The urging member 36a is disposed on a side opposite to the rotary plate 32 with the first conduit switching plate 33 interposed between the urging member 36a and the rotary plate 32.

The urging member 36a urges the first conduit switching plate 33 toward the rotary plate 32.

The urging member 36b is disposed on a side opposite to the rotary plate 32 with the second conduit switching plate 34 interposed between the urging member 36b and the rotary plate 32.

The urging member 36b urges the second conduit switching plate 34 toward the rotary plate 32.

The urging member 36c is disposed on a side opposite to the rotary plate 32 with the third conduit switching plate 35 interposed between the urging member 36c and the rotary plate 32.

The urging member 36c urges the third conduit switching plate 35 toward the rotary plate 32.

By rotating the rotary plate 32 about the axis of rotation O, the cam 32a moves any one of the first conduit switching plate 33, the second conduit switching plate 34, and the third conduit switching plate 35 in a direction away from the rotary plate 32 in the direction of the axis of rotation O, thus selecting whether communication of the first conduit 2b1 is enabled, communication of the second conduit 2b2 is enabled, and communication of the air suction conduit 2k is enabled.

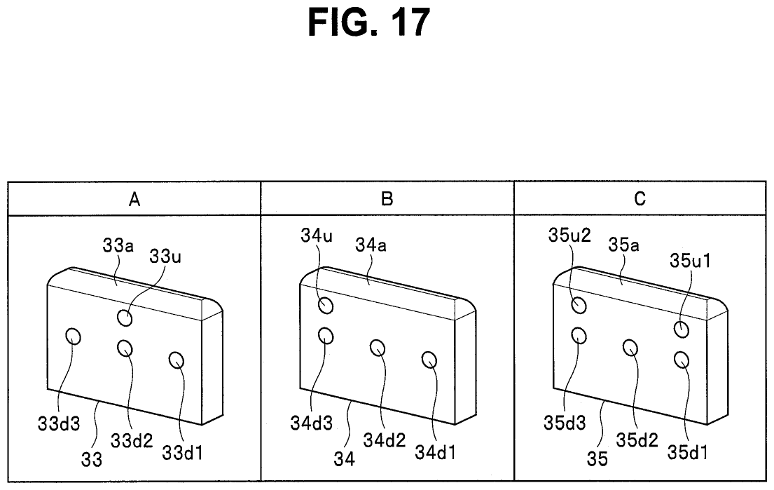

FIG. 17 is a table showing configuration examples of the first conduit switching plate 33, the second conduit switching plate 34, and the third conduit switching plate 35 of the conduit selection device 2 of the eighth embodiment as viewed in an oblique direction.

Column A in FIG. 17 shows the first conduit switching plate 33, column B in FIG. 17 shows the second conduit switching plate 34, and column C in FIG. 17 shows the third conduit switching plate 35.

As shown in columns A to C in FIG. 17, a cam receiver 33a is provided at one end side of the first conduit switching plate 33 that faces the rotary plate 32, a cam receiver 34a is provided at one end side of the second conduit switching plate 34 that faces the rotary plate 32, and a cam receiver 35a is provided at one end side of the third conduit switching plate 35 that faces the rotary plate 32.

In an example shown in FIG. 17, the cam receivers 33a, 34a, 35a also have a cam shape. The switching mechanism 2f is configured to be switched between the first state (column A), the second state (column C), and the third state (column B). In the third state, the first conduit 2b1 is prevented from communicating with the common conduit 2b3. In the third state, the second conduit 2b2 is at a third pressure, the third pressure is less than the second pressure of the second conduit 2b2 in the second state. In the third state, the first conduit 2b1 cannot be in communication with the common conduit, the second conduit 2b2 cannot be in communication with the common conduit, and the second conduit 2b2 can suction from outside of the body 2a.

However, either the cam 32a or the cam receivers 33a, 34a, 35a may have a cam shape.

Providing a cam to at least either the cam 32a or the cam receivers 33a, 34a, 35a allows the first conduit switching plate 33, the second conduit switching plate 34, and the third conduit switching plate 35 to be smoothly moved in the direction of the axis of rotation O by the rotary plate 32.

The first conduit switching plate 33 has an upper communication hole and lower communication holes, the second conduit switching plate 34 has an upper communication hole and lower communication holes, and the third conduit switching plate 35 has upper communication holes and lower communication holes.

The upper communication hole and the lower communication holes form portions of the branch conduits 2b.

The upper communication hole is formed at a position close to the rotary plate 32 in the direction of the axis of rotation O, and the lower communication holes are formed at positions away from the rotary plate 32 in the direction of the axis of rotation O.

The upper communication hole or the upper communication holes are holes that communicate with one or more conduits selected from the first conduit 2b1, the second conduit 2b2, and the air suction conduit 2k, when the first conduit switching plate 33, the second conduit switching plate 34, or the third conduit switching plate 35 is in a depressed position in which the first conduit switching plate 33, the second conduit switching plate 34, or the third conduit switching plate 35 is brought into contact with the cam 32a, thus being moved downward against an urging force of the urging member 36a, 36b, or 36c.

The lower communication holes are holes that communicate with one or more conduits selected from the first conduit 2b1, the second conduit 2b2, and the air suction conduit 2k, when each of the first conduit switching plate 33, the second conduit switching plate 34, and the third conduit switching plate 35 is in a standard position in which the first conduit switching plate 33, the second conduit switching plate 34, or the third conduit switching plate 35 is brought into no contact with the cam 32a, thus not being moved downward.

As shown in column A, the first conduit switching plate 33 has a communication hole 33u (second communication hole) at an upper portion of the first conduit switching plate 33, and has three communication holes 33d1, 33d2, 33d3 at a lower portion of the first conduit switching plate 33.

The communication hole 33u communicates with the second conduit 2b2 when the first conduit switching plate 33 is in a depressed position.

The communication holes 33d1, 33d2, 33d3 respectively communicate with the first conduit 2b1, the second conduit 2b2, and the air suction conduit 2k when the first conduit switching plate 33 is in a standard position.

As shown in column B, the second conduit switching plate 34 has a communication hole 34u at an upper portion of the second conduit switching plate 34, and has three communication holes 34d1, 34d2, 34d3 at a lower portion of the second conduit switching plate 34.

The communication hole 34u communicates with the air suction conduit 2k when the second conduit switching plate 34 is in a depressed position.

The communication holes 34d1, 34d2, 34d3 respectively communicate with the first conduit 2b1, the second conduit 2b2, and the air suction conduit 2k when the second conduit switching plate 34 is in a standard position.

As shown in column C, the third conduit switching plate 35 has two communication holes 35u1, 35u2 at an upper portion of the third conduit switching plate 35, and has three communication holes 35d1, 35d2, 35d3 at a lower portion of the third conduit switching plate 35.

The communication holes 35u1, 35u2 respectively communicate with the first conduit 2b1 and the air suction conduit 2k when the third conduit switching plate 35 is in a depressed position.

The communication holes 35d1, 35d2, 35d3 respectively communicate with the first conduit 2b1, the second conduit 2b2, and the air suction conduit 2k when the third conduit switching plate 35 is in a standard position.

When the rotary plate 32 is rotated to a position at which the index mark 32c points to the liquid feeding index mark 2m1, the first conduit switching plate 33 and the second conduit switching plate 34 take a standard position, but the third conduit switching plate 35 is depressed by the cam 32a, thus taking a depressed position.

With such an operation, communication of the first conduit 2b1 is established via the communication hole 33d1, the communication hole 34d1, and the communication hole 35u1.

Further, communication of the air suction conduit 2k is established via the communication hole 33d3, the communication hole 34d3, and the communication hole 35u2.

In contrast, the second conduit 2b2 faces the third conduit switching plate 35 at a portion that has no communication hole, thus being closed by the third conduit switching plate 35.

The first state (liquid feeding: on, suction: off) is achieved in this manner.

In this state, air can be suctioned through the air suction conduit 2k even when the suction device 8 is in actuation and hence, it is possible to reduce a load on the suction conduit.

When the rotary plate 32 is rotated to a position at which the index mark 32c points to the suction index mark 2m2, the second conduit switching plate 34 and the third conduit switching plate 35 take a standard position, but the first conduit switching plate 33 is depressed by the cam 32a, thus taking a depressed position.

With such an operation, communication of the second conduit 2b2 is established via the communication hole 33u, the communication hole 34d2, and the communication hole 35d2.

In contrast, the first conduit 2b1 and the air suction conduit 2k face portions of the first conduit switching plate 33 that have no communication hole, thus being closed by the first conduit switching plate 33.

The second state (liquid feeding: off, suction: on) is achieved in this manner.

When the rotary plate 32 is rotated to a position at which the index mark 32c points to the off index mark 2m3a or the off index mark 2m3b, the first conduit switching plate 33 and the third conduit switching plate 35 take a standard position, but the second conduit switching plate 34 is depressed by the cam 32a, thus taking a depressed position.

With such an operation, communication of the air suction conduit 2k is established via the communication hole 33d3, the communication hole 34u, and the communication hole 35d3.

In contrast, the first conduit 2b1 and the second conduit 2b2 face portions of the second conduit switching plate 34 that have no communication hole, thus being closed by the second conduit switching plate 34. The switching mechanism 2f can include the first conduit switching plate 33 and the second conduit switching plate 34, the first conduit switching plate 33 has the first communication hole 35u1, the second conduit switching plate has the second communication hole 33u. In the first state, the first conduit 2b1 communicates with the common conduit 2b3 via the first hole 35u1, and in the second state, the second conduit 2b2 communicates with the second conduit via the second hole 33u.

The third state (liquid feeding: off, suction: off) is achieved in this manner.

In this state, air can be suctioned through the air suction conduit 2k even when the suction device 8 is in actuation and hence, it is possible to reduce a load on the suction conduit.

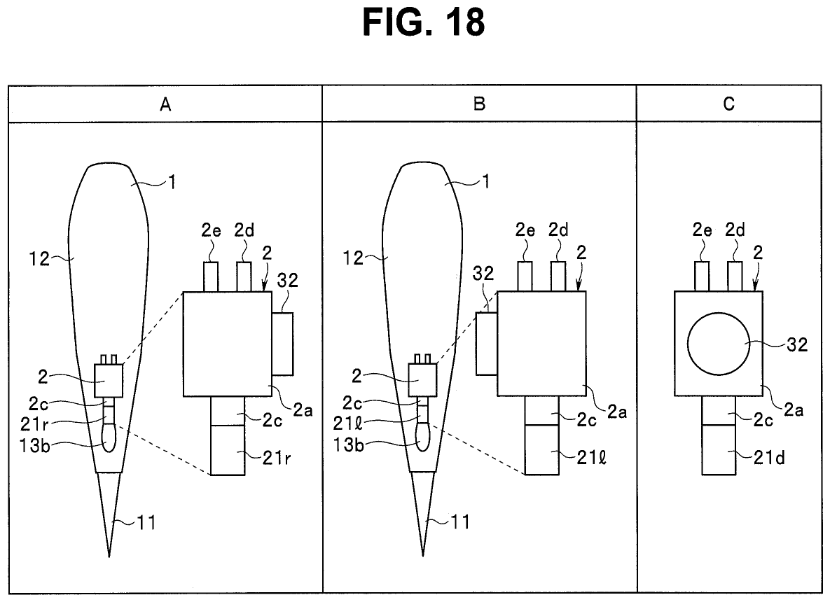

FIG. 18 is a table showing configuration examples of a conduit selection device 2 of a modification of the eighth embodiment.

As described in the second embodiment, the conduit selection device 2 may be connected to the port forming the proximal-end-side opening 13b of the endoscope 1 with the joint part 21.

Column A in FIG. 18 shows an example of a right handed joint part 21r.

In the conduit selection device 2 that is connected to the port forming the proximal-end-side opening 13b with the right handed joint part 21r, the rotary plate 32 is disposed on a right side of the conduit selection device 2.

Column B in FIG. 18 shows an example of the left handed joint part 211.

In the conduit selection device 2 that is connected to the port forming the proximal-end-side opening 13b with the left handed joint part 211, the rotary plate 32 is disposed on a left side of the conduit selection device 2.

When the rotary plate 32 is disposed on a side in a direction opposite to a direction toward a dominant hand as shown in columns A, B in FIG. 18, the operator can easily operate the rotary plate 32.

Column C in FIG. 18 shows an example of a right-handed and left-handed common joint part 21d.

In the conduit selection device 2 that is connected to the port forming the proximal-end-side opening 13b with the common joint part 21d, the rotary plate 32 is disposed on a side opposite to the operation portion 12.

By using the joint part 21d as shown in column C in FIG. 18, an endoscope can be used by the operator irrespective of the dominant hand of the operator.

According to the eighth embodiment having such a configuration, it is possible to obtain advantageous effects substantially the same as the advantageous effects of the above-described first to seventh embodiments.

Assume a case in which the second conduit switching plate 34 is disposed on one side of the first conduit switching plate 33 or on one side of the third conduit switching plate 35, the first conduit switching plate 33 and the third conduit switching plate 35 being arranged in parallel. In such a case, when the conduit selection device 2 is shifted from the first state to the third state (or from the third state to the first state), the conduit selection device 2 may be shifted via the second state, or when the conduit selection device 2 is shifted from the second state to the third state (or from the third state to the second state), the conduit selection device 2 may be shifted via the first state. Accordingly, ease of operation is low.

In contrast, in the eighth embodiment, the second conduit switching plate 34 is disposed between the first conduit switching plate 33 and the third conduit switching plate 35. Accordingly, in a case in which the conduit selection device 2 is in the first state or the second state, even when the rotary plate 32 is rotated in either the right rotation direction or the left rotation direction, the conduit selection device 2 can be directly shifted to the third state and hence, excellent ease of operation can be achieved.

### Ninth Embodiment

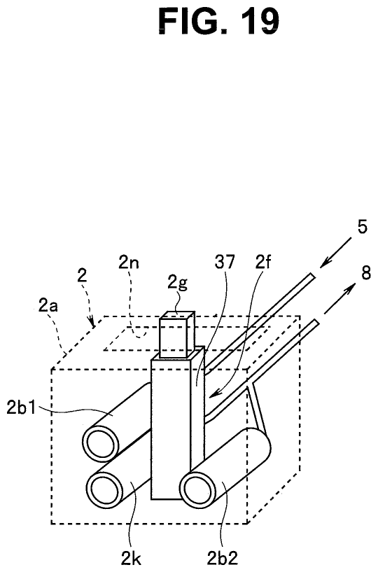

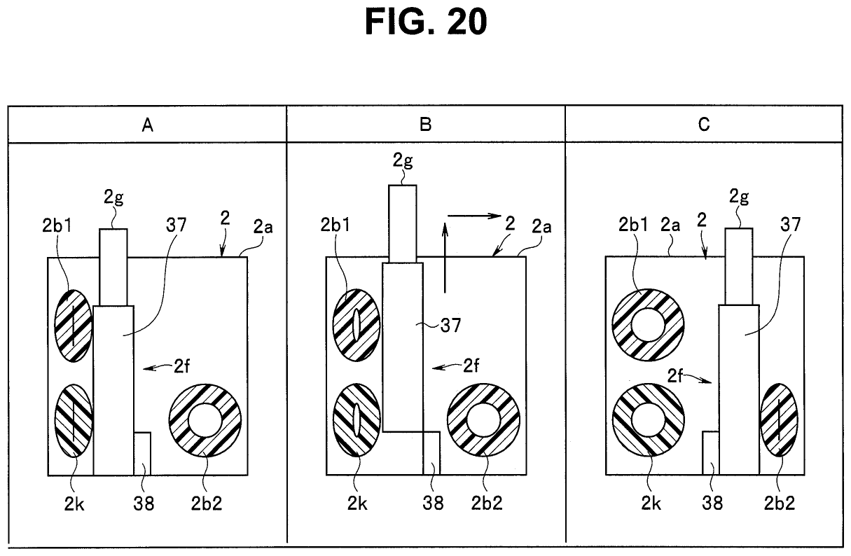

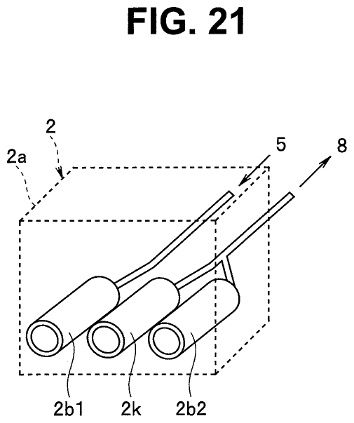

FIG. 19 and FIG. 20 show a ninth embodiment of the present disclosure.

FIG. 19 is a diagram showing a configuration example of a conduit selection device 2 of the ninth embodiment.

FIG. 20 is a table illustrating a state of switching the conduit selection device 2 of the ninth embodiment between the first state and the second state.

In the ninth embodiment, components substantially the same as the corresponding components in the first to eighth embodiments are given the same reference symbols and the description of such components will be omitted when appropriate, and points that make the ninth embodiment different from the first to eighth embodiments will be mainly described.

As shown in FIG. 19, a slide hole 2n extending in a sliding direction is formed in the body 2a of the conduit selection device 2.

A slider 37 forming the selection mechanism 2f is provided in the body 2a.

The slider 37 is movable in the sliding direction.

The operation device 2g is provided at an end portion of the slider 37.

The operation device 2g protrudes to the outside of the body 2a from the slide hole 2n, thus allowing the operator to operate the operation device 2g.

As shown in FIGS. 19 to 27, in the first state, the switching mechanism 2f is configured to compress the second conduit 2b2 without compressing the first conduit 2b1. In the second state, the switching mechanism 2f is configured to compress the first conduit 2b1 without compressing the second conduit 2b2.

In some embodiments, the switching mechanism 2f can include a rotor 32 or 51 rotatable about a rotation axis with 32 or 51 to the body 2a to switch between the first state and the second state. The rotor 32 can include a plate 32b or 5 lb and a protrusion 32a, 51a or 51b extending from the plate 32b or 51b. In the first state, the protrusion 32a, 51a or 51b is configured to compress the second conduit 2b2, and in the second state, compress the first conduit 2b1.

The first conduit 2b1 and the air suction conduit 2k are disposed in the body 2a along an inner wall of the body 2a at one end side in the sliding direction, and the second conduit 2b2 is disposed in the body 2a along an inner wall of the body 2a at the other end side in the sliding direction.

In the respective embodiments following the ninth embodiment, the first conduit 2b1, the second conduit 2b2, and the air suction conduit 2k are made of a material having elasticity, and can be pressed and compressed (that is, each conduit can be closed).

A stopper 38 protruding upward from a bottom surface in the body 2a is provided at a center portion of the bottom surface in the sliding direction.

A length of the slider 37 (a portion excluding the operation device 2g) is shorter than a distance between an upper end of the stopper 38 and a ceiling surface in the body 2a.

The slider 37 is further movable in an up and down direction in FIG. 20 from a position, at which a lower end of the slider 37 comes into contact with the bottom surface in the body 2a, to a position, at which an upper end of the slider 37 comes into contact with the ceiling surface in the body 2a.

Accordingly, when the slider 37 is moved in the upward direction in FIG. 20, the slider 37 can cross over the stopper 38 and can move in the sliding direction.

Column A in FIG. 20 shows the second state (liquid feeding: off, suction: on).

The lower end of the slider 37 is in contact with the bottom surface in the body 2a at a position between the first conduit 2b1/the air suction conduit 2k and the stopper 38.

In this state, the slider 37 serves as a press shape unit that selectively presses and compresses the first conduit 2b1 and the air suction conduit 2k between the slider 37 and inner wall of the body 2a.

The stopper 38 restricts a position of the slider 37 in the sliding direction to prevent the slider 37 from being pushed back by a reaction force due to elasticity of the first conduit 2b1 and the air suction conduit 2k.

The second conduit 2b2 is not compressed by the slider 37, and communication of the second conduit 2b2 is established.

Therefore, the second state is selected.

Column C in FIG. 20 shows the first state (liquid feeding: on, suction: off).

The lower end of the slider 37 is in contact with the bottom surface in the body 2a at a position between the second conduit 2b2 and the stopper 38.

In this state, the slider 37 serves as the press shape unit that selectively presses and compresses the second conduit 2b2 between the slider 37 and the inner wall of the body 2a.

The stopper 38 restricts the position of the slider 37 in the sliding direction to prevent the slider 37 from being pushed back by a reaction force due to elasticity of the second conduit 2b2.

The first conduit 2b1 and the air suction conduit 2k are not compressed by the slider 37, and communication of the first conduit 2b1 and communication of the air suction conduit 2k are established.

Therefore, the first state is selected.

In this state, air can be suctioned through the air suction conduit 2k even when the suction device 8 is in actuation and hence, it is possible to reduce a load on the suction conduit.

Column B in FIG. 20 shows a state in the course of switching from the second state shown in column A to the first state shown in column C.

When the slider 37 is pulled up by the operation device 2g in the upward direction in FIG. 20 to a height that causes the slider 37 to cross over the stopper 38, and is then slid, the second state is switched to the first state or the second state is switched to the first state.

Although not shown in the drawing, as a modification of the ninth embodiment, protrusions may be provided in the slide hole 2n in place of the stopper 38.

The protrusions provided in the slide hole 2n hold the operation device 2g at a first position or a second position.

When the operation device 2g is held at the first position, the slider 37 selects the first state.

When the operation device 2g is held at the second position, the slider 37 selects the second state.

Click sensing may be generated when the first state is selected.

With such a configuration, the operator can surely perceive selection of the first state.

Click sensing may be generated when the second state is selected.

With such a configuration, the operator can surely perceive selection of the second state.

According to the ninth embodiment having such a configuration, it is possible to obtain advantageous effects substantially the same as the advantageous effects of the above-described first to eighth embodiments.

In addition to the above, liquid feeding and suction are switched by selectively pressing and compressing the first conduit 2b1, the second conduit 2b2, and the air suction conduit 2k made of a material having elasticity and hence, it is possible to reduce the number of components, thus lowering manufacturing costs.

### Tenth Embodiment

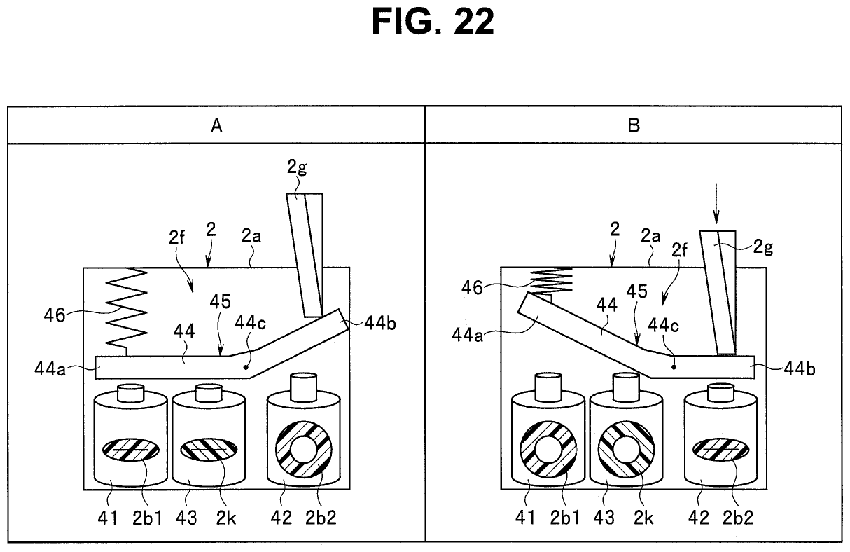

FIG. 21 and FIG. 22 show a tenth embodiment of the present disclosure.

FIG. 21 is a diagram showing an example of arrangement of the first conduit 2b1, the second conduit 2b2, and the air suction conduit 2k in the conduit selection device 2 of the tenth embodiment.

FIG. 22 is a table showing a configuration example of the conduit selection device 2 of the tenth embodiment.

In the tenth embodiment, components substantially the same as the corresponding components in the first to ninth embodiments are given the same reference symbols and the description of such components will be omitted when appropriate, and points that make the tenth embodiment different from the first to ninth embodiments will be mainly described.

As shown in FIG. 21, in the body 2a of the conduit selection device 2, the first conduit 2b1, the air suction conduit 2k, and the second conduit 2b2 are arranged in this order.

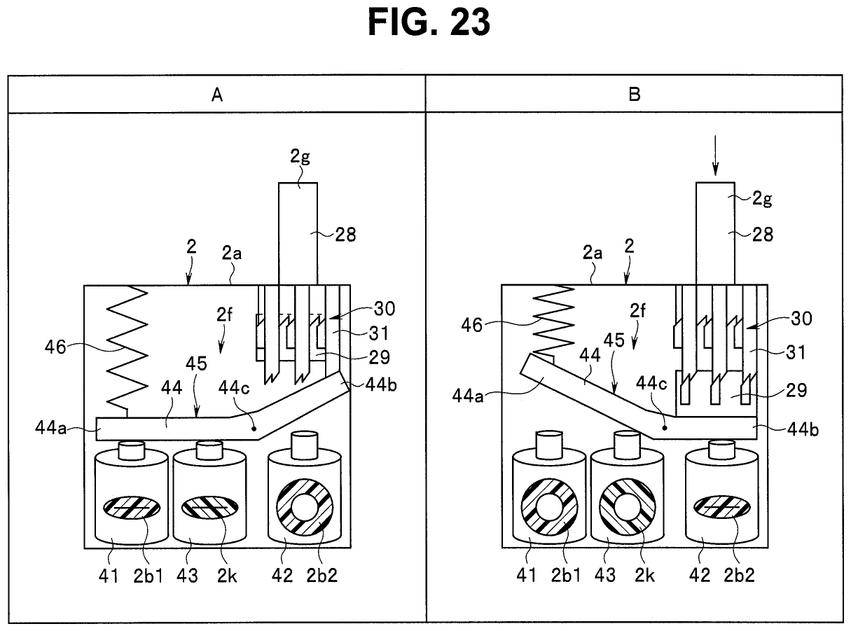

As shown in FIG. 22, a selection mechanism 2f of the conduit selection device 2 is configured to include a seesaw mechanism 45 (lever). As shown in FIGS. 22 and 23, the switching mechanism 2f can includes the input controller 2g configured to receive the external force, and the lever 45 configured to receive the external force from the input controller 2g, and to compress one of the first conduit 2b1, the second conduit 2b2, and the third conduit 2k to switch between the first state and the second state.

In the body 2a, a pinch valve 41 is provided on the first conduit 2b1, a pinch valve 43 is provided on the air suction conduit 2k, and a pinch valve 42 is provided on the second conduit 2b2.

In the body 2a, a lever 44 is provided as a constituent element of the seesaw mechanism 45.

The lever 44 is swingably supported by a fulcrum 44c provided between the pinch valve 43 and the pinch valve 42.

The fulcrum 44c is formed of a pin or the like that swingably fixes the lever 44 to the body 2a.

One arm portion 44a of the lever 44 faces the pinch valve 41 and the pinch valve 43 in such a way as to be capable of pressing the pinch valve 41 and the pinch valve 43, and the other arm portion 44b of the lever 44 faces the pinch valve 42 in such a way as to be capable of pressing the pinch valve 42.

An urging member 46 is attached to the arm portion 44a.

The urging member 46 urges the arm portion 44a in a direction in which the pinch valve 41 and the pinch valve 43 are pressed.

The arm portion 44b can be pressed by the operation device 2g formed of a button or the like that receives an external force (pressing force).

When the operation device 2g is pressed against an urging force of the urging member 46, the arm portion 44b presses the pinch valve 43.

When the operation device 2g, which is formed of a button or the like, receives an external force and is moved, the operation device 2g presses the arm portion 44b on one end side of the seesaw mechanism 45, thus causing the seesaw mechanism 45 to be inclined.

When the seesaw mechanism 45 is inclined, one of the plurality of states including the first state and the second state is selected.

Column A in FIG. 22 shows the second state (liquid feeding: off, suction: on).

When the operation device 2g does not press the arm portion 44b, the arm portion 44a presses the pinch valve 41 and the pinch valve 43 by the urging force of the urging member 46.

The first conduit 2b1 and the air suction conduit 2k are pressed and compressed by the pinch valve 41 and the pinch valve 43.

The pinch valve 42 is not pressed and hence, the second conduit 2b2 is not compressed and communication of the second conduit 2b2 is established.

Column B in FIG. 22 shows the first state (liquid feeding: on, suction: off).

When the operation device 2g is pressed against the urging force of the urging member 46, the arm portion 44b presses the pinch valve 42.

The second conduit 2b2 is pressed and compressed by the pinch valve 42.

The pinch valve 41 and the pinch valve 43 are not pressed and hence, the first conduit 2b1 and the air suction conduit 2k are not compressed and communication of the first conduit 2b1 and communication of the air suction conduit 2k are established.

In this state, air can be suctioned through the air suction conduit 2k even when the suction device 8 is in actuation and hence, it is possible to reduce a load on the suction conduit.

The above-described lever 44 and pinch valves 41, 42, 43 form a press shape unit that selectively presses and compresses the first conduit 2b1/the air suction conduit 2k and the second conduit 2b2.

According to the tenth embodiment having such a configuration, it is possible to obtain advantageous effects substantially the same as the advantageous effects of the above-described first to ninth embodiments.

### Eleventh Embodiment

FIG. 23 shows an eleventh embodiment of the present disclosure.

FIG. 23 is a table showing a configuration example of a conduit selection device 2 of the eleventh embodiment.

In the eleventh embodiment, components substantially the same as the corresponding components in the first to tenth embodiments are given the same reference symbols and the description of such components will be omitted when appropriate, and points that make the eleventh embodiment different from the first to tenth embodiments will be mainly described.

A selection mechanism 2f of the conduit selection device 2 includes, in addition to the above-described seesaw mechanism 45, the knock cam mechanism 30 including the button 28 equipped with cams, the rotor 29, the rib 31 and the like.

A detailed configuration and a manner of operation of the knock cam mechanism are substantially same as those described in the sixth embodiment.

Column A in FIG. 23 shows the second state (liquid feeding: off, suction: on).

In this state, the rotor 29 is not pressed in the sliding direction and hence, the arm portion 44a of the lever 44 presses the pinch valve 41 and the pinch valve 43 by the urging force of the urging member 46.

The first conduit 2b1 and the air suction conduit 2k are pressed and compressed by the pinch valve 41 and the pinch valve 43.

Column B in FIG. 23 shows the first state (liquid feeding: on, suction: off).

In this state, the rotor 29 is moved in the sliding direction and hence, the arm portion 44b pressed by the rotor 29 presses the pinch valve 42.

The second conduit 2b2 is pressed and compressed by the pinch valve 42.

At this point of operation, the rotor 29 engages with the cams of the ribs 31, so that a position of the rotor 29 in the sliding direction is held.

In the same manner as described above, a load on the suction conduit is reduced.

The first state shown in column B in FIG. 23 and the second state shown in column A in FIG. 23 can be switched each time the button 28 of the knock cam mechanism 30 is pressed one time.

According to the eleventh embodiment having such a configuration, it is possible to obtain advantageous effects substantially the same as the advantageous effects of the above-described first to tenth embodiments.

In the tenth embodiment, although the second state is held by the urging force of the urging member 46, to hold the first state, the operation device 2g may be continued pressing.

In contrast, in the eleventh embodiment, there is an advantage that after the conduit selection device 2 is switched to either the first state or the second state by pressing the button 28 one time, the first state or the second state is held.

### Twelfth Embodiment

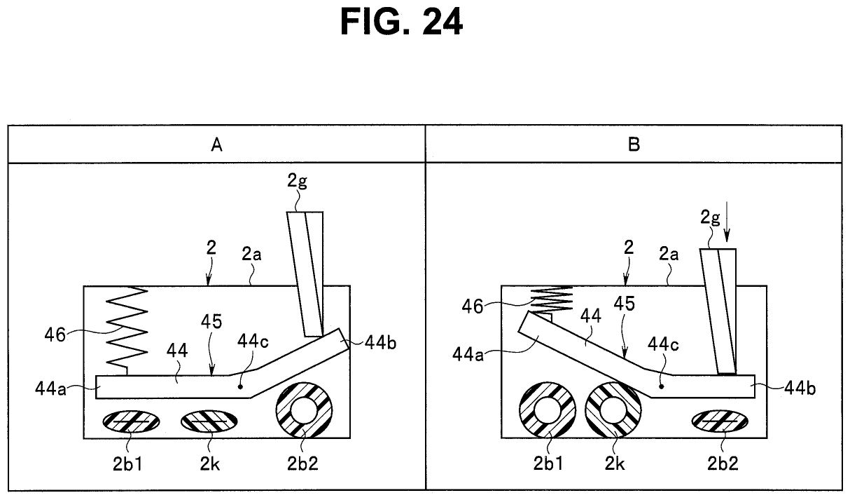

FIG. 24 shows a twelfth embodiment of the present disclosure.

FIG. 24 is a table showing a configuration example of a conduit selection device 2 of the twelfth embodiment.

In the twelfth embodiment, components substantially the same as the corresponding components in the first to eleventh embodiments are given the same reference symbols and the description of such components will be omitted when appropriate, and points that make the twelfth embodiment different from the first to eleventh embodiments will be mainly described.

The conduit selection device 2 of the twelfth embodiment is obtained by omitting the pinch valves 41, 42, 43 from the conduit selection device 2 of the tenth embodiment.

The first conduit 2b1 and the air suction conduit 2k are directly pressed by the arm portion 44a of the lever 44 of the seesaw mechanism 45.

The second conduit 2b2 is directly pressed by the arm portion 44b of the lever 44.

Column A in FIG. 24 shows the second state (liquid feeding: off, suction: on).

In this state, the arm portion 44a that receives the urging force of the urging member 46 directly presses and compresses the first conduit 2b1 and the air suction conduit 2k.

Column B in FIG. 24 shows the first state (liquid feeding: on, suction: off).

In this state, the arm portion 44b that receives a pressing force of the operation device 2g directly presses and compresses the second conduit 2b2.

In the same manner as described above, a load on the suction conduit is reduced.

The above-described arm portion 44a and arm portion 44b of the lever 44 form a press shape unit that selectively presses and compresses the first conduit 2b1/the air suction conduit 2k and the second conduit 2b2.

According to the twelfth embodiment having such a configuration, it is possible to obtain advantageous effects substantially the same as the advantageous effects of the above-described first to eleventh embodiments.

In addition to the above, the number of parts is smaller than the number of parts in the tenth embodiment and the eleventh embodiment and hence, it is possible to reduce manufacturing costs.

### Thirteenth Embodiment

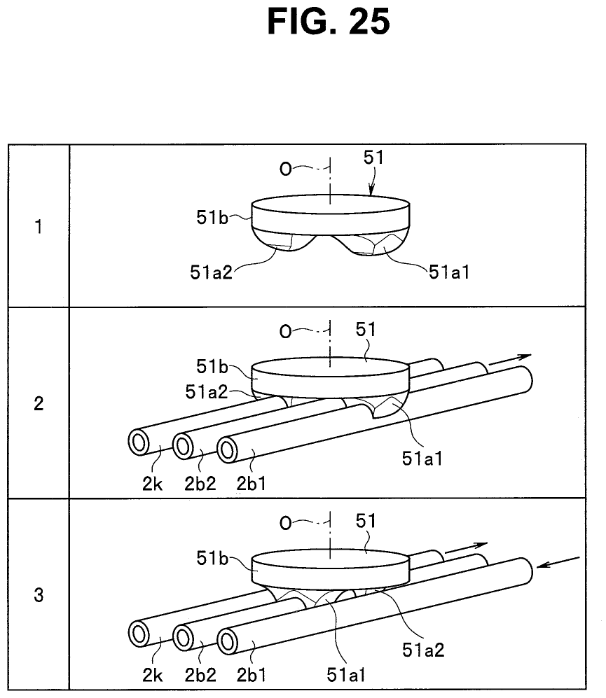

FIG. 25 shows a thirteenth embodiment of the present disclosure.

FIG. 25 is a table showing a configuration example of a conduit selection device 2 of the thirteenth embodiment.

In the thirteenth embodiment, components substantially the same as the corresponding components in the first to twelfth embodiments are given the same reference symbols and the description of such components will be omitted when appropriate, and points that make the thirteenth embodiment different from the first to twelfth embodiments will be mainly described.

A selection mechanism 2f includes a rotary plate 51.

The rotary plate 51 is a rotor that rotates with respect to the body 2a about an axis of rotation O.

The selection mechanism 2f selects one from the plurality of states including the first state and the second state according to a rotation angle of the rotary plate 51.

As shown in row 1 in FIG. 25, the rotary plate 51 is a cam-equipped disk in which a first cam 51a1 and a second cam 51a2 each having unevenness are provided on one surface of the cam-equipped disk in the direction of the axis of rotation O.

The first cam 51a1 and the second cam 51a2 are provided on the rotary plate 51 at positions opposite to each other in the circumferential direction (positions differ from each other by 180 degrees), and have shapes that are symmetrical with respect to the axis of rotation O.

As shown in row 2 and row 3 in FIG. 25, the rotary plate 51 has the surface provided with the cams 51a1, 51a2.

The surface provided with the cams 51a1, 51a2 is disposed in such a way as to face the first conduit 2b1, the second conduit 2b2, and the air suction conduit 2k.

The cams 51a1, 51a2 form a press shape unit that selectively presses and compresses one or more conduits selected from the first conduit 2b1, the second conduit 2b2, and the air suction conduit 2k.

In the body 2a of the conduit selection device 2, the first conduit 2b1, the second conduit 2b2, and the air suction conduit 2k are arranged in this order.

A peripheral surface 51b of the rotary plate 51 having a disk shape may be knurled so as to facilitate a rotation operation of the rotary plate 51.

Alternatively, the rotation operation of the rotary plate 51 may be facilitated by covering the peripheral surface 51b of the rotary plate 51 and a surface of the rotary plate 51 on a side opposite to the cams 51a1, 51a2 with a rubber cap or the like that can generate friction.

Row 2 in FIG. 25 shows the second state (liquid feeding: off, suction: on).

In this state, one of the cams 51a1, 51a2 presses and compresses the first conduit 2b1, and the other of the cams 51a1, 51a2 presses and compresses the air suction conduit 2k.

The second conduit 2b2 comes into contact with neither the cam 51a1 nor the cam 51a2 and hence, the second conduit 2b2 is not compressed and communication of the second conduit 2b2 is established.

Row 3 in FIG. 25 shows the first state (liquid feeding: on, suction: off).

In this state, both the cams 51a1, 51a2 press and compress the second conduit 2b2.

The first conduit 2b1 and the air suction conduit 2k come into contact with neither the cam 51a1 nor the cam 51a2 and hence, the first conduit 2b1 and the air suction conduit 2k are not compressed and communication of the first conduit 2b1 and communication of the air suction conduit 2k are established.

Accordingly, air can be suctioned through the air suction conduit 2k even when the suction device 8 is maintained to be actuated at the time of performing liquid feeding and hence, it is possible to reduce a load on the suction conduit.

The first state and the second state are switched each time the rotary plate 51 is rotated by 90 degrees in any rotational direction about the axis of rotation O.

According to the thirteenth embodiment having such a configuration, it is possible to obtain advantageous effects substantially the same as the advantageous effects of the above-described first to twelfth embodiments.

In addition to the above, even when the rotary plate 51 is rotated in either the right rotation direction or the left rotation direction, the first state and the second state can be switched and hence, excellent ease of operation can be achieved.

### Fourteenth Embodiment

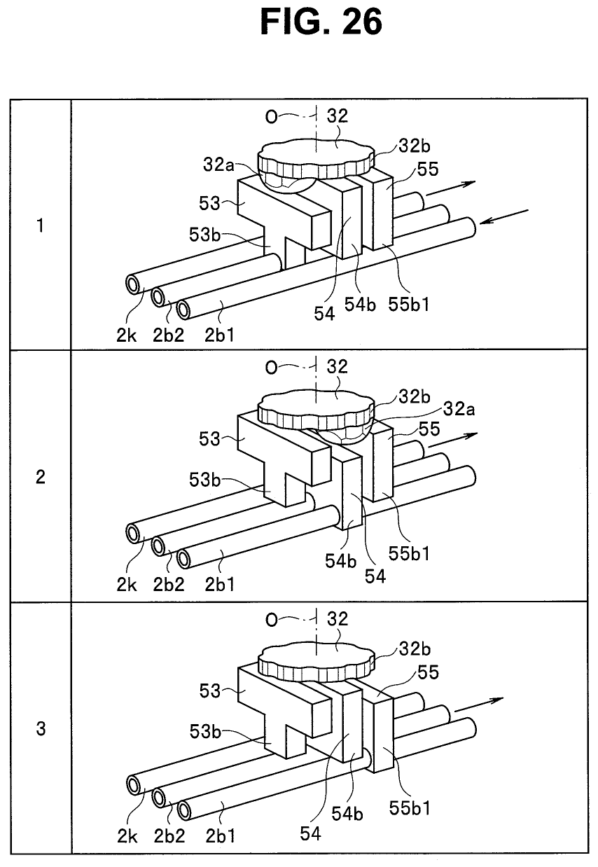

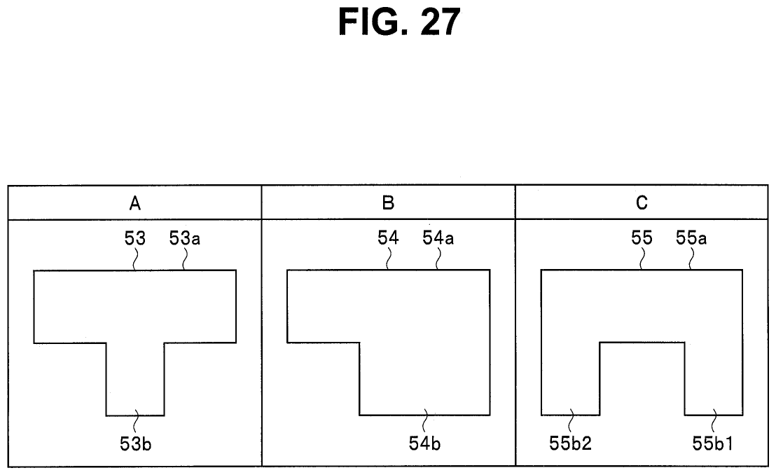

FIG. 26 and FIG. 27 show a fourteenth embodiment of the present disclosure.

FIG. 26 is a table showing a configuration example of a conduit selection device 2 of the fourteenth embodiment.

In the fourteenth embodiment, components substantially the same as the corresponding components in the first to thirteenth embodiments are given the same reference symbols and the description of such components will be omitted when appropriate, and points that make the fourteenth embodiment different from the first to thirteenth embodiments will be mainly described.

A selection mechanism 2f includes the rotary plate 32, a first conduit switching plate 53 (porting body), a second conduit switching plate 54, and a third conduit switching plate 55.

In the same manner as the first conduit switching plate 33, the second conduit switching plate 34, and the third conduit switching plate 35 shown in FIG. 14, the first conduit switching plate 53, the second conduit switching plate 54, and the third conduit switching plate 55 are respectively urged by the urging members 36a, 36b, 36c toward the rotary plate 32. However, the illustration of the urging members 36a, 36b, 36c is omitted.

The switching mechanism 2f includes the porting body 53 slidably movable relative to the body 2a between a first position and a second position, and an input controller configured to receive an external force and to translate the external force to the porting body to switch the porting body between the first position and the second position. The input controller can be the rotary plate 32, button, lever, dial, etc.

A configuration of the rotary plate 32 is basically the same as the configuration of rotary plate described in the eighth embodiment.

As shown in FIG. 16, the index mark 32c is provided on a surface of the rotary plate 32, and the liquid feeding index mark 2m1, the off index mark 2m3a, the suction index mark 2m2, and the off index mark 2m3b are provided on a surface of the body 2a at positions around the rotary plate 32.

The first conduit switching plate 53, the second conduit switching plate 54, and the third conduit switching plate 55 are disposed in such a way as to be movable in the direction of the axis of rotation O.

By rotating the rotary plate 32 about the axis of rotation O, the cam 32a moves any one of the first conduit switching plate 53, the second conduit switching plate 54, and the third conduit switching plate 55 in a direction away from the rotary plate 32 in the direction of the axis of rotation O.

Such an operation selects whether communication of the first conduit 2b1 is enabled, communication of the second conduit 2b2 is enabled, and communication of the air suction conduit 2k is enabled.

FIG. 27 is a table showing configuration examples of the first conduit switching plate 53, the second conduit switching plate 54, and the third conduit switching plate 55 of the conduit selection device 2 of the fourteenth embodiment.

Column A in FIG. 27 shows the first conduit switching plate 53, column B in FIG. 27 shows the second conduit switching plate 54, and column C in FIG. 27 shows the third conduit switching plate 55.

As shown in columns A to C in FIG. 27, a cam receiver 53a is provided at one end side of the first conduit switching plate 53 that faces the rotary plate 32, a cam receiver 54a is provided at one end side of the second conduit switching plate 54 that faces the rotary plate 32, and a cam receiver 55a is provided at one end side of the third conduit switching plate 55 that faces the rotary plate 32.

In the same manner as the cam receivers 33a, 34a, 35a shown in FIG. 17, the cam receivers 53a, 54a, 55a may have a cam shape.

As shown in column A in FIG. 27, the first conduit switching plate 53 is provided with a protruding portion 53b that protrudes toward the second conduit 2b2.

As shown in column B in FIG. 27, the second conduit switching plate 54 is provided with a protruding portion 54b that protrudes toward the first conduit 2b1 and the second conduit 2b2.

As shown in column C in FIG. 27, the third conduit switching plate 55 is provided with a first protruding portion 55b1 that protrudes toward the first conduit 2b1 and with a second protruding portion 55b2 that protrudes toward the air suction conduit 2k.

The protruding portion 53b, the protruding portion 54b, the first protruding portion 55b1, and the second protruding portion 55b2 form a press shape unit that selectively presses and compresses one or more conduits selected from the first conduit 2b1, the second conduit 2b2, and the air suction conduit 2k.

When each of the first conduit switching plate 53, the second conduit switching plate 54, and the third conduit switching plate 55 is brought into no contact with the cam 32a, each of the first conduit switching plate 53, the second conduit switching plate 54, and the third conduit switching plate 55 takes a standard position in which the first conduit switching plate 53, the second conduit switching plate 54, or the third conduit switching plate 55 is not moved downward. When each of the first conduit switching plate 53, the second conduit switching plate 54, and the third conduit switching plate 55 is brought into contact with the cam 32a, each of the first conduit switching plate 53, the second conduit switching plate 54, and the third conduit switching plate 55 is moved downward, thus taking a depressed position.

When each of the first conduit switching plate 53, the second conduit switching plate 54, and the third conduit switching plate 55 is in the standard position, each of the first conduit switching plate 53, the second conduit switching plate 54, and the third conduit switching plate 55 presses none of the first conduit 2b1, the second conduit 2b2, and the air suction conduit 2k.

When the rotary plate 32 is rotated to a position at which the index mark 32c points to the liquid feeding index mark 2m1, as shown in row 1 in FIG. 26, the first conduit switching plate 53 takes a depressed position, and the second conduit switching plate 54 and the third conduit switching plate 55 take a standard position.

In such a state, the protruding portion 53b of the first conduit switching plate 53 presses and compresses the second conduit 2b2, but neither the first conduit 2b1 nor the air suction conduit 2k is compressed and communication of the first conduit 2b1 and communication of the air suction conduit 2k are established.

The first state (liquid feeding: on, suction: off) is achieved in this manner.

In this state, air can be suctioned through the air suction conduit 2k even when the suction device 8 is in actuation and hence, it is possible to reduce a load on the suction conduit.

When the rotary plate 32 is rotated to a position at which the index mark 32c points to the off index mark 2m3a or the off index mark 2m3b, as shown in row 2 in FIG. 26, the second conduit switching plate 54 takes a depressed position, and the first conduit switching plate 53 and the third conduit switching plate 55 take a standard position.

In such a state, the protruding portion 54b of the second conduit switching plate 54 presses and compresses the first conduit 2b1 and the second conduit 2b2, but the air suction conduit 2k is not compressed and communication of the air suction conduit 2k is established.

The third state (liquid feeding: off, suction: off) is achieved in this manner.

In this state, air can be suctioned through the air suction conduit 2k even when the suction device 8 is in actuation and hence, it is possible to reduce a load on the suction conduit.

When the rotary plate 32 is rotated to a position at which the index mark 32c points to the suction index mark 2m2, as shown in row 3 in FIG. 26, the third conduit switching plate 55 takes a depressed position, and the first conduit switching plate 53 and the second conduit switching plate 54 take a standard position.

In such a state, the first protruding portion 55b1 of the third conduit switching plate 55 presses and compresses the first conduit 2b1 and the second protruding portion of the third conduit switching plate 55 presses and compresses the air suction conduit 2k, but the second conduit 2b2 is not compressed and communication of the second conduit 2b2 is established.

The second state (liquid feeding: off, suction: on) is achieved in this manner. The switching mechanism 2f is configured to be switched to between the first state (row 1), the second state (row 3), and the third state (row 2). In the third state, the first conduit 2b1 is prevented from communicating with the common conduit 2b3. In the third state, the second conduit 2b2 is at a third pressure, the third pressure is less than the second pressure of the second conduit 2b2 in the second state. In the third state, the first conduit 2b1 cannot be in communication with the common conduit, the second conduit 2b2 cannot be in communication with the common conduit, and the second conduit 2b2 can suction from outside of the body 2a.

Note that, in the same manner as the eighth embodiment, the conduit selection device 2 may be connected to the port forming the proximal-end-side opening 13b of the endoscope 1 with the right handed joint part 21r shown in column A in FIG. 18, the left handed joint part 211 shown in column B in FIG. 18, or the right handed and left handed common joint part 21d shown in column C in FIG. 18.

According to the fourteenth embodiment having such a configuration, it is possible to obtain advantageous effects substantially the same as the advantageous effects of the above-described first to thirteenth embodiments.

The second conduit switching plate 54 is disposed between the first conduit switching plate 53 and the third conduit switching plate 55. Accordingly, in a case in which the conduit selection device 2 is in the first state or the second state, even when the rotary plate 32 is rotated in either the right rotation direction or the left rotation direction, the conduit selection device 2 can be directly shifted to the third state and hence, excellent ease of operation can be achieved in the same manner as the eighth embodiment.

The present disclosure is not limited to the above-described embodiments as it is, and can be embodied by applying a modification to constituent elements in the implementation stage without departing from the gist of the present disclosure.

Further, various modes of the disclosure can be formed by suitably combining a plurality of constituent elements disclosed in the embodiments.

For example, some constituent elements of all constituent elements described in the embodiments may be deleted.

The constituent elements used in different embodiments may be suitably combined.

As described above, needless to say, various modifications and applications are conceivable without departing from the gist of the disclosure.
Example 1. A conduit selection device comprising:
   a connection port configured to be detachably mounted on an endoscope and to communicate with an end portion of an endoscope conduit;
   a branch conduit having one end side and another end side, the one end side communicating with the connection port, the other end side branching into a first conduit and a second conduit;
   a fluid supply port communicating with the first conduit and configured to allow a fluid flowing into the conduit selection device from outside to flow to the endoscope conduit via the first conduit;
   a fluid outflow port communicating with the second conduit and configured to allow a fluid flowing out from the endoscope conduit to flow to a suction device via the second conduit, the suction device being provided outside; and
   a selection mechanism attached to the branch conduit and configured to select whether the connection port is enabled to communicate with either the fluid supply port or the fluid outflow port.
Example 2. The conduit selection device according to Example 1, wherein
   the selection mechanism selects one from a plurality of states including
      a first state in which communication is established between the connection port and the fluid supply port, but no communication is established between the connection port and the fluid outflow port, and
      a second state in which no communication is established between the connection port and the fluid supply port, but communication is established between the connection port and the fluid outflow port.
Example 3. The conduit selection device according to Example 2, further comprising
   a pressure regulating mechanism configured to reduce a negative pressure in the second conduit caused by the suction device in a case in which the first state is selected by the selection mechanism.
Example 4. The conduit selection device according to Example 3, wherein
   the pressure regulating mechanism includes a mechanism configured, in the case in which the first state is selected by the selection mechanism, to switch the second conduit such that communication is established between the fluid outflow port and an external atmosphere.
Example 5. The conduit selection device according to Example 4, wherein
   the first state is a state in which communication is established between the connection port and the fluid supply port, no communication is established between the connection port and the fluid outflow port, and communication is established between the fluid outflow port and the external atmosphere.
Example 6. The conduit selection device according to Example 5, wherein
   the selection mechanism selects one from a plurality of states including the first state, the second state, and a third state in which no communication is established between the connection port and the fluid supply port and no communication is established between the connection port and the fluid outflow port, but communication is established between the fluid outflow port and the external atmosphere.
Example 7. The conduit selection device according to Example 3, wherein
   the pressure regulating mechanism includes a switch configured to stop the suction device in the case in which the first state is selected by the selection mechanism.
Example 8. The conduit selection device according to Example 7, further comprising:
   a body housing at least a portion of the branch conduit, wherein
   the selection mechanism includes a rotor configured to rotate with respect to the body about an axis of rotation, and selects one from the plurality of states including the first state and the second state according to a rotation angle of the rotor, and
   the switch is attached to the body, and actuates or stops the suction device according to the rotation angle of the rotor.
Example 9. The conduit selection device according to Example 8, wherein
   the switch stops the suction device in a case in which the rotor is at a rotation angle at which the first state is selected, and the switch actuates the suction device in a case in which the rotor is at a rotation angle at which the second state is selected.
Example 10. The conduit selection device according to Example 2, wherein
   the first conduit and the second conduit are made of a material having elasticity,
   the selection mechanism includes a press shape unit configured to selectively press and compress the first conduit and the second conduit, and
   the first state is selected in a case in which the press shape unit compresses the second conduit without compressing the first conduit, and the second state is selected in a case in which the press shape unit compresses the first conduit without compressing the second conduit.
Example 11. The conduit selection device according to Example 2, further comprising
   a body housing at least a portion of the branch conduit, wherein
   the selection mechanism includes a rotor configured to rotate with respect to the body about an axis of rotation, and selects one from the plurality of states including the first state and the second state according to a rotation angle of the rotor.
Example 12. The conduit selection device according to Example 11, wherein
   the rotor is a rotary plate in which an unevenness is provided on one surface of the rotary plate in a direction of the axis of rotation,
   the rotary plate is disposed such that the unevenness faces the first conduit and the second conduit, and
   by rotating the rotary plate about the axis of rotation, the unevenness selects whether communication of the first conduit is enabled and communication of the second conduit is enabled.
Example 13. The conduit selection device according to Example 12, wherein
   the selection mechanism further includes a conduit switching plate having one end side and another end side in the direction of the axis of rotation, the one end side facing the unevenness, the other end side facing an intermediate portion of the first conduit and an intermediate portion of the second conduit, and
   by rotating the rotary plate about the axis of rotation, the unevenness moves the conduit switching plate in the direction of the axis of rotation, thus selecting whether communication of the first conduit is enabled and communication of the second conduit is enabled.
Example 14. The conduit selection device according to Example 2, further comprising
   a body housing at least a portion of the branch conduit, wherein
   the selection mechanism includes a slide mechanism including at least another portion of the branch conduit, and configured to slide in a case in which the slide mechanism receives an external force, and
   one of the plurality of states including the first state and the second state is selected according to a slide position of the slide mechanism.
Example 15. The conduit selection device according to Example 14, wherein
   the slide mechanism includes a slide body and a button, the slide body housing at least the other portion of the branch conduit, the button being configured to slide the slide body in a case in which the button receives the external force, and
   the slide mechanism selects one from the plurality of states including the first state and the second state according to a position of the slide body.
Example 16. The conduit selection device according to Example 15, wherein
   the selection mechanism includes a knock cam mechanism including
      an urging member configured to urge the slide body toward the button,
      a rotor disposed between the slide body and the button, and
      cams provided on the body, the button, and the rotor, and
   each time the button receives the external force, the cams changing a position of the slide body via the rotor to switch between the first state and the second state.
Example 17. The conduit selection device according to Example 2, further comprising:
   a body housing at least a portion of the branch conduit, wherein
   the selection mechanism includes
      a button configured to receive an external force, and
      a seesaw mechanism configured to be swingably supported on the body by a fulcrum, and
   in a case in which the button receives the external force and is moved, the button presses one end side of the seesaw mechanism, thus causing the seesaw mechanism to be inclined to select one from the plurality of states including the first state and the second state.
Example 18. An endoscope apparatus comprising:
   an endoscope including an insertion portion, an operation portion, and an endoscope conduit provided in at least the insertion portion;
   a suction device provided outside the endoscope, and configured to suction a fluid; and
   a conduit selection device, wherein
   the conduit selection device includes
   a connection port configured to be detachably mounted on the endoscope and to communicate with an end portion of the endoscope conduit,
   a branch conduit having one end side and another end side, the one end side communicating with the connection port, the other end side branching into a first conduit and a second conduit,
   a fluid supply port communicating with the first conduit and configured to allow a fluid flowing into the conduit selection device from the outside to flow to the endoscope conduit via the first conduit,
   a fluid outflow port communicating with the second conduit and configured to allow a fluid flowing out from the endoscope conduit to flow to the suction device via the second conduit, and
   a selection mechanism attached to the branch conduit and configured to select whether the connection port is enabled to communicate with either the fluid supply port or the fluid outflow port.
Example 19. The endoscope apparatus according to Example 18, wherein
   the conduit selection device is positioned by the connection port at a position outside the endoscope and adjacent to the operation portion.
Example 20. The endoscope apparatus according to Example 18, further comprising
   a pressure regulating mechanism configured to reduce a negative pressure in the second conduit caused by the suction device in a case in which a state in which no communication is established between the connection port and the fluid outflow port is selected by the selection mechanism.
Example 21. The endoscope apparatus according to Example 18, wherein
   the first conduit and the second conduit are made of a material having elasticity,
   the selection mechanism includes a press shape unit configured to selectively press and compress the first conduit and the second conduit, and
   in a case in which the press shape unit compresses the second conduit without compressing the first conduit, a first state is selected in which communication is established between the connection port and the fluid supply port, but no communication is established between the connection port and the fluid outflow port, and
   in a case in which the press shape unit compresses the first conduit without compressing the second conduit, a second state is selected in which no communication is established between the connection port and the fluid supply port, but communication is established between the connection port and the fluid outflow port.
Example 22. The endoscope apparatus according to Example 18, wherein
   the endoscope is a single-use endoscope that is disposed of after being used one time.
Example 23. A processor, the processor is configured to:
   receive one of a first signal or a second signal from a sensor,
   actuate a suction pump to perform suction when the first signal is received, and
   stop the suction pump to prevent suction when the second signal is received.
Example 24. The conduit selection device according to Example 1, a negative pressure occurred inside of the second conduit is suppressed or reduced.
Example 25. The conduit selection device according to Example 1, wherein, in the first state, the second conduit is at a first pressure,
   wherein, in the second state, the second conduit is at a second pressure, and
   wherein the first pressure is less than the second pressure.
Example 25. The conduit selection device according to Example 25, wherein the switching mechanism is further configured to be switched to a third state,
   wherein, in the third state, the second conduit is at a third pressure,
   wherein the third pressure is less than the second pressure.

What is claimed is:

1. A conduit switching device, comprising:
a body;
an endoscope connection port;
a fluid supply port;
a fluid suction port;
a first conduit in the body and the fluid supply port;
a second conduit in the body and the fluid suction port;
a common conduit in the body and the endoscope connection port; and
a switching mechanism configured to be switched between a first state and a second state;
wherein in the first state, the first conduit communicates with the common conduit, and the second conduit does not communicate with the common conduit,
wherein in the second state, the first conduit does not communicate with the common conduit, and the second conduit communicates with the common conduit.

2. The conduit switching device according to claim 1, wherein the switching mechanism is further configured to be switched to a third state, and
wherein, in the third state, the first conduit does not communicate with the common conduit.

3. The conduit switching device according to claim 1, wherein a part of the second conduit has a sealing member to prevent a fluid inside of the second conduit from leaking to outside of the second conduit.

4. The conduit switching device according to claim 1, wherein in the first state, the second conduit suctions from an outside of the body.

5. The conduit switching device according to claim 4, wherein the switching mechanism includes:
a porting body slidably movable relative to the body between a first position and a second position, and
an input controller configured to receive an external force and to translate the external force to the porting body to switch the porting body between the first position and the second position.

6. The conduit switching device according to claim 5, wherein the switching mechanism further includes:
a biasing element configured to bias the porting body toward the input controller, and
a knock cam mechanism between the input controller and the porting body, and
wherein the knock cam mechanism is configured to translate the external force from the input controller to the porting body to switch the porting body between the first position and the second position.

7. The conduit switching device according to claim 1, wherein the switching mechanism is further configured to be switched to a third state, and
wherein, in the third state:
the first conduit does not be in communication with the common conduit,
the second conduit does not be communication with the common conduit, and
the second conduit suctions from outside of the body.

8. The conduit switching device according to claim 1, further comprising a sensor,
wherein the sensor is configured to:
detect one of the first state and the second state,
generate a first signal when the first state is detected, and
generate a second signal when the second state is detected,
wherein the first signal actuates a suction device, and
wherein the second signal prevents actuation of the suction device.

9. The conduit switching device according to claim 1, further comprising:
a sensor, and
a processor,
wherein the sensor is configured to:
detect one of the first state and the second state,
generate a first signal when the first state is detected, and
generate a second signal when the second state is detected, and
wherein the processor is configured to:
receive one of the first signal or the second signal from the sensor, and
actuate the suction device to perform suction when the first signal is received and stop the suction device to prevent suction when the second signal is received.

10. The conduit switching device according to claim 9, wherein the switching mechanism further includes a rotor,
wherein the rotor rotates with respect to the body about a longitudinal axis of the body, and
wherein the sensor generates one of the first signal and the second signal in response to a rotation angle of the rotor.

11. The conduit switching device according to claim 10, wherein, in the first state, the rotation angle is within a first angle range,
wherein, in the second state, the rotation angle is within a second angle range, and
wherein the second angle range is smaller than the first angle range.

12. The conduit switching device according to claim 1, wherein the switching mechanism is configured to
in the first state, compress the second conduit without compressing the first conduit, and
in the second state, compress the first conduit without compressing the second conduit.

13. The conduit switching device according to claim 1, wherein the switching mechanism includes a rotor rotatable about a rotation axis with respect to the body to switch between the first state and the second state.

14. The conduit switching device according to claim 13, wherein the rotor includes a plate and a protrusion extending from the plate, and
wherein the protrusion is configured to:
in the first state, compress the second conduit, and
in the second state, compress the first conduit.

15. The conduit switching device according to claim 1, wherein the switching mechanism further includes a first conduit switching plate and a second conduit switching plate,
wherein the first conduit switching plate has a first communication hole,
wherein the second conduit switching plate has a second communication hole,
wherein, in the first state, the first conduit communicates with the common conduit via the first hole, and
wherein, in the second state, the second conduit communicates with the second conduit via the second hole.

16. The conduit switching device according to claim 1, further comprising a third conduit,
wherein the third conduit communicates between the second conduit and an outside of the body, and
wherein the switching mechanism is configured to:
in the first state, suction fluid from the outside of the body via the third conduit, and prevent the second conduit from suctioning fluid from the common conduit, and
in the second state, suction fluid from the common conduit via the second conduit, and prevent the third conduit from suctioning fluid from the outside of the body.

17. The conduit switching device according to claim 16, wherein the switching mechanism includes:
an input controller configured to receive an external force, and
a lever configured to receive the external force from the input controller, and to compress one of the first conduit, the second conduit, and the third conduit to switch between the first state and the second state.

18. An endoscope system, comprising:
the conduit switching device according to claim 1,
an endoscope including an insertion portion, an operation portion, and an endoscope conduit provided in at least the insertion portion; and
a suction device provided outside the endoscope.

19. The endoscope system according to claim 18, wherein the endoscope is connected to the endoscope connection port of the conduit switching device with the endoscope conduit in communication with common conduit.

20. The endoscope system according to claim 18, wherein the endoscope is a single-use endoscope.
